(19) United States
(12) Patent Application Publication
(10) Pub. No.: US 2017/0114001 A1
(43) Pub. Date: Apr. 27, 2017

(54) PROCESS FOR PREPARING ALKANOLAMINES USEFUL IN REMOVAL OF ACID-GAS FROM A GASEOUS STREAM

(71) Applicant: The Queen's University of Belfast, Belfast, Antrim (GB)

(72) Inventors: Martin Atkins, Belfast, Antrim (GB); Fergal Coleman, Belfast, Antrim (GB); Yoan Delavoux, Belfast, Antrim (GB)

(21) Appl. No.: 15/129,445

(22) PCT Filed: Mar. 27, 2015
(86) PCT No.: PCT/GB2015/050952

§ 371 Date: Sep. 27, 2016

(30) Foreign Application Priority Data
Mar. 27, 2014 (GB) 1405563.6

(51) Int. Cl. C07C 213/02; C07D 301/32; C07C 209/80; F23J 15/02; C07D 207/06; C07C 209/84; B01J 31/02; B01D 53/14; C07D 301/02; C07C 68/00

(52) U.S. Cl. CPC C07C 213/02; C07D 301/02; C07D 301/32; C07C 209/80; C07C 68/00; C07D 207/06; C07C 209/84; B01J 31/0282; B01D 53/1462; B01D 53/1493; F23J 15/02; C07C 2523/00; B01D 2252/20489

(57) ABSTRACT

The invention relates to a process for preparing alkanolamines, useful in the removal of CO₂ and/or H₂S from a CO₂ and/or H₂S containing gaseous stream, wherein the preparation of the alkanolamines is conducted using specifically selected ionic liquids under specifically selected reaction conditions.

The invention relates to a process for preparing alkanolamines useful in the removal of CO₂ and/or H₂S from a CO₂ and/or H₂S containing gaseous stream. More specifically, the invention relates to a process where the preparation of the alkanolamines is conducted using specifically selected ionic liquids using specifically selected reaction conditions.

The presence of CO₂ and H₂S acidic (“sour”) gases in gaseous streams is a well-known issue for oil refineries and chemical and gas processing plants. In most cases, it is necessary to incorporate additional processing steps in order to remove acid-gases from gaseous streams thus allowing those streams to have commercial utility, for example as a fuel source. Several “sweetening” processes for removing CO₂ and/or H₂S from gaseous streams are known.

One such process involves absorption of CO₂ and/or H₂S with alkanolamine based solvents. Commonly, an alkanolamine is provided in the form of an aqueous solution which reacts exothermically, and reversibly, when brought into contact with the acidic-gases in the gaseous stream.

The typical reactions of aqueous secondary and tertiary alkanolamines with CO₂ and H₂S can be represented as follows:

H₂S+R₃N [symbol] R₃NH⁺+SH⁻ (1)

H₂S+R₂NH [symbol] R₂NH₂⁺+SH⁻ (2)

CO₂+2R₂NH [symbol] R₂NH₂⁺+R₂NCOO⁻ (3)

CO₂+R₃N+H₂O [symbol] R₃NH⁺+HCO₃⁻ (4)

With respect to CO₂ absorption, only primary and secondary alkanolamines can form a carbamate directly from the reaction with CO₂, as illustrated in the reactions below.

CO₂+R₂NH+H₂O [symbol] R₂NCOO⁻+H₃O⁺ (3)(i)

R₂NCOO⁻+H₃O⁺ [symbol] R₂NCOOH+H₂O (3)(ii)

The formation of carbamate by primary and secondary alkanolamines is known to limit the molar CO₂ absorption capacity of aqueous alkanolamine solutions. This arises because carbamate formation consumes two moles of alkanolamine for every mole of CO₂, as illustrated in equilibrium reaction (3) above. Thus, the molar absorption capacity of an alkanolamine is limited on the basis of whether it is a primary, secondary or tertiary alkanolamine. Furthermore, absorption capacity is also affected by the basicity of the amine functionality of the alkanolamine. Generally, as the pKa of the amine functionality of the alkanolamine increases, CO₂ absorption capacity also increases.

It is not possible to form a carbamate directly from reaction of CO₂ with tertiary alkanolamines, which lack the necessary proton. Tertiary alkanolamines instead lead to the formation of a carbonate anion when reacted with CO₂ in the presence of water. In such an aqueous system, a tertiary alkanolamine acts as a sink for hydrogen ions produced when CO₂ hydrolyses in water, as illustrated in the reactions below.

CO₂+H₂O [symbol] H⁺+HCO₃⁻ (4)(i)

HCO₃⁻ [symbol] H⁺+CO₃⁻ (4)(ii)

R₃N+H⁺ [symbol] R₃NH⁺ (4)(iii)

Although a number of alkanolamines, and mixtures thereof, have been suggested for gas sweetening in the past, only a small number have been found to be commercially viable. These include monoethanolamine (MEA), diethanolamine (DEA), methyldiethanolamine (MDEA), diisopropanolamine (DIPA) and diglycolamine (DGA). This is likely to be a result of the economic impact of their use. For instance, use of aqueous amines in CO₂ capture is known to consume almost 30% of the energy that is needed to run a power plant (Fisher et al., “Advanced amine solvent formulations and process integration for near-term CO₂ capture success”, Trimeric Corporation, Pennsylvania, 2007).

Although absorption capacity is an important consideration for alkanolamine based absorbents, rate of CO₂ absorption is also a key factor. Slow CO₂ absorption rates lead to longer gas-liquid contact times requiring larger absorption columns and greater capital costs. There is thus often a compromise between the benefits of having high absorption capacity versus the negative impact of lower absorption kinetics. This is particularly relevant to the use of MDEA as an acid-gas absorbent. As a tertiary amine, MDEA has a higher absorption capacity than MEA. However, its rate of absorption is so low that its absorption capacity is rarely realised in commercially viable systems. It is for this reason that MDEA is commonly utilised in conjunction with an activator, such as piperazine, as disclosed in U.S. Pat. No. 4,336,233. WO 95/03874 also discloses the use of an aqueous mixture of MDEA and methylmonoethanolamine (MMEA), resulting in the in situ generation of N,N′-bis(dimethyl)-N-hydroxyethyl-ethylenediamine (DMHEED), which is said to improve acid-gas removal.

The kinetics involved in the recycle of alkanolamine, i.e. desorption of acid-gas, also represents an important economic consideration in the overall sweetening process. For instance, regeneration of primary and secondary alkanolamines from the corresponding carbamates often proceeds via boiling of the aqueous solution (thereby stripping CO₂) followed by cooling and recycle of the alkanolamine, which is energy intensive. High desorption rates are thus desirable to reduce the energy expended in regenerating alkanolamine on an industrial scale. On a related point, losses of volatile lower boiling point alkanolamines, often as a result of the desorption process which can be conducted at temperatures well in excess of 100° C., have affected the choice of alkanolamine in industry. For instance, it is for this reason that MEA, with a boiling point of 170° C., was superseded by DEA, having a much higher boiling point of 280° C., as the alkanolamine of choice for mitigating alkanolamine losses. MDEA, with a boiling point of 247° C., also benefits from a lower volatility than MEA. Absorbents having high boiling points and low vapour pressure are thus generally preferred.

Yet a further issue affecting the economic viability of the use of alkanolamine absorbents for CO₂ and/or H₂S removal from gaseous streams is the cost of the bulk materials required for preparing the desired alkanolamines. For instance, industry standards MEA, DEA and MDEA are prepared from ethylene oxide with ammonia, the former being a particularly expensive starting material. Meanwhile, as MDEA is most commonly used in the form of piperazine activated MDEA, its cost is increased further, partly as a result of piperazine being expensive to make on a large scale. Use of piperazine activated MDEA is also not environmentally friendly and there are restrictions relating to the transport of MDEA. A further issue affecting the economics of gas sweetening with alkanolamine absorbents is that the alkanolamines are sold on a weight basis, but utilised in an industrial capacity on a volume basis. Often the weight to volume ratio of alkanolamines impacts negatively on the economics of their use.

There remains a need for alternative and economically advantageous alkanolamine absorbents for use in acid-gas capture, specifically there remains a need for a process which is capable of preparing useful alkanolamines efficiently, using low cost materials.

The present invention is based on the surprising discovery that alkanolamines prepared from the reaction of glycerol (GL), dimethyl carbonate (DMC) and various amines are particularly useful absorbents for acid-gas capture. In particular, an advantageous process has been found for preparing useful alkanolamines, comprising the use of ionic liquids under specifically selected reaction conditions.

GL is produced in large quantities as a by-product in the production of biodiesels. With an increasing focus on the use of biofuels to at least partly replace petroleum fuels, the production of GL has increased to levels far higher than current demand. As a result, GL is cheap and readily available, particularly in countries where production of biofuels is prevalent. Moreover, since GL may be derived from biomass it can be considered to be a green material. DMC is also considered to be a green reagent (see Kreutzberger., Charles B. (2001), “Chloroformates and Carbonates”, Kirk-Othmer Encyclopedia of Chemical Technology, New York: John Wiley) and is also relatively cheap, particularly in comparison to ethylene oxide which is used in the preparation of known alkanolamine absorbents, such as industry standards MEA, DEA and MDEA.

In a first aspect, the present invention provides a process for preparing an alkanolamine compound of formula I and/or an alkanolamine compound of formula II, or salts thereof:

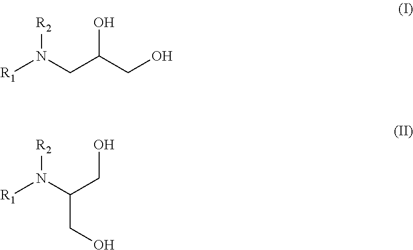

wherein: R₁ and R₂ are independently selected from hydrogen, a C₁ to C₈, straight chain or branched alkyl group, a C₂ to C₈ straight chain or branched alkenyl group, a C₃ to C₈ cycloalkyl group, a C₆ to C₁₀ aryl group, a 3 to 10 membered heterocyclic group, a polysaccharide group, a polyethylene oxide group, or R₁ and R₂ together with the nitrogen atom to which they are attached form a heterocyclic group, wherein said alkyl, alkenyl, cycloalkyl, aryl and heterocyclic groups are unsubstituted or may be substituted by one to three groups selected from: C₁ to C₆ alkoxy, C₂ to C₈ alkoxyalkoxy, C₃ to C₈ cycloalkyl, C₆ to C₁₀ aryl, 3 to 10 membered heterocyclic, C₇ to C₁₀ aralkyl, 3 to 10 membered heterocyclic-C₁ to C₄ alkyl, —OH, —CH₂CH(OH)CH₂(OH), —CH(CH₂OH)₂, or —NR₃R₄, wherein R₃ and R₄ are independently selected from hydrogen or C₁ to C₆ straight chain or branched alkyl group;
   said process comprising the following steps:
i) preparing glycidol by a one-pot reaction of glycerol with dimethylcarbonate, at a temperature of from 100° C. to 160° C. and a molar ratio of glycerol to dimethylcarbonate of from 1:4 to 1:10, in the presence of an ionic liquid catalyst having the formula:

[Cat⁺][X⁻]
wherein: [Cat⁺] represents one or more cationic species, and
   [X⁻] represents one or more anionic species;
ii) reacting the product of step i) with an amine of formula III,

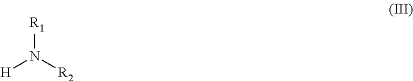

wherein: R₁ and R₂ are as defined for formula I and II.

The term “aryl” used herein, either alone or as part of another group, refers to an aromatic cyclic or fused bicyclic hydrocarbon group. Examples of aryl groups as referred to herein include phenyl and naphthyl groups. A preferred aryl group is phenyl.

The term “cycloalkyl” used herein refers to a non-aromatic cyclic hydrocarbon group. Examples of a cycloalkyl group as referred to herein include cyclopropyl, cyclobutyl, cyclopentyl, and cyclohexyl. A preferred cycloalkyl group is cyclopropyl.

The term “heterocyclic” used herein refers to an aromatic or non-aromatic cyclic or fused bicyclic hydrocarbon group comprising one or more of O, N, NH and S in the ring. Examples of a heterocyclic group as referred to herein include ethylene oxide, azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, azepinyl, pyridyl, pyrazinyl, pyrimidinyl, pyridazinyl, triazinyl, triazolyl, tetrazolyl, tetrahydropyranyl, morpholinyl, thiamorpholinyl. Preferred heterocyclic compounds include pyrrolyl, pyrrolidinyl and piperidinyl.

The term “catalyst” as used herein refers to a substance which increases the rate of a chemical reaction without itself being consumed by the reaction. In particular, the ionic liquid catalyst used increases the rate of transesterification between glycerol and dimethylcarbonate to form glycerol carbonate and/or increases the rate of decarboxylation of glycerol carbonate to form glycidol.

The term “ionic liquid” as used herein refers to a liquid that is capable of being produced by melting a salt, and when so produced consists solely of ions. An ionic liquid may be formed from a homogeneous substance comprising one species of cation and one species of anion, or it can be composed of more than one species of cation and/or more than one species of anion. Thus, an ionic liquid may be composed of more than one species of cation and one species of anion. An ionic liquid may further be composed of one species of cation, and one or more species of anion. Still further, an ionic liquid may be composed of more than one species of cation and more than one species of anion.

The term “ionic liquid” includes compounds having both high melting points and compounds having low melting points, e.g. at or below room temperature. Thus, many ionic liquids have melting points below 200° C., particularly below 100° C., around room temperature (15 to 30° C.), or even below 0° C. Ionic liquids having melting points below around 30° C. are commonly referred to as “room temperature ionic liquids” and are often derived from organic salts having nitrogen-containing heterocyclic cations. In room temperature ionic liquids, the structures of the cation and anion prevent the formation of an ordered crystalline structure and therefore the salt is liquid at room temperature.

Ionic liquids are most widely known as solvents. Many ionic liquids have been shown to have negligible vapour pressure, temperature stability, low flammability and recyclability. Due to the vast number of anion/cation combinations that are available it is possible to fine-tune the physical properties of the ionic liquid (e.g. melting point, density, viscosity, and miscibility with water or organic solvents) to suit the requirements of a particular application.

In accordance with the present invention, [Cat⁺] may comprise a cationic species selected from: ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium, and uronium.

In one preferred embodiment of the invention, [Cat⁺] comprises an acyclic cation selected from:

[N(R^{a})(R^{b})(R^{c})(R^{d})]⁺, [P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₃₀, straight chain or branched alkyl group, a C₃ to C₈ cycloalkyl group, or a C₆ to C₁₀ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

More preferably, [Cat⁺] comprises a cation selected from:

[N(R^{a})(R^{b})(R^{c})(R^{d})]⁺, [P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₁₅ straight chain or branched alkyl group, a C₃ to C₆ cycloalkyl group, or a C₆ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

Further examples include wherein R^{a}, R^{b}, R^{c} and R^{d} are independently selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl. More preferably two or more, and most preferably three or more, of R^{a}, R^{b}, R^{c} and R^{d} are selected from methyl, ethyl, propyl and butyl.

Still more preferably, [Cat⁺] comprises a cation selected from:

[N(R^{a})(R^{b})(R^{c})(R^{d})]⁺,
wherein: R^{a}, R^{b}, R^{c}, and R^{d} are as defined above.

In a preferred further embodiment, [Cat⁺] preferably comprises a cation selected from:

[P(R^{a})(R^{b})(R^{c})(R^{d})]⁺,
wherein: R^{a}, R^{b}, R^{c}, and R^{d} are as defined above.

Specific examples of preferred ammonium and phosphonium cations suitable for use according to the present invention include:

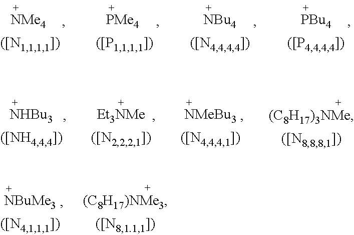

Specific examples of more preferred ammonium cations suitable for use according to the present invention include:

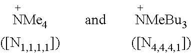

In a further embodiment of the invention, [Cat⁺] comprises a cation selected from guanidinium, cyclic guanidinium, uronium, cyclic uronium, thiuronium and cyclic thiuronium. More preferably, [Cat⁺] comprises a cation having the formula:

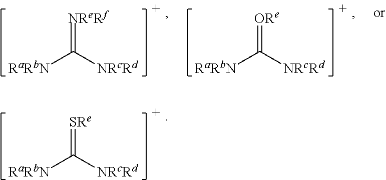

wherein: R^{a}, R^{b}, R^{c}, R^{d}, R^{e}, and R^{f} are each independently selected from a C₁ to C₃₀, straight chain or branched alkyl group, a C₃ to C₈ cycloalkyl group, or a C₆ to C₁₀ aryl group, or any two of R^{a}, R^{b}, R^{c}, and R^{d}, attached to different nitrogen atoms form a methylene chain —(CH₂)_{q}— wherein q is from 2 to 5; wherein said alkyl, cycloalkyl or aryl groups or said methylene chain are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

Specific examples of guanidinium, uronium, and thiuronium cations suitable for use according to the present invention include:

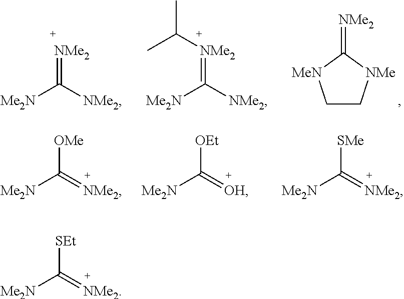

In a further preferred embodiment, [Cat⁺] comprises a cation comprising an electron-rich sulfur or selenium moiety. Examples include cations as defined above comprising pendant thiol, thioether, or disulfide substituents.

In another embodiment of the invention, [Cat⁺] comprises an aromatic heterocyclic cationic species selected from: benzimidazolium, benzofuranium, benzothiophenium, benzotriazolium, cinnolinium, diazabicyclodecenium, diazabicyclononenium, diazabicyclo-undecenium, dithiazolium, imidazolium, indazolium, indolinium, indolium, oxazinium, oxazolium, iso-oxazolium, oxathiazolium, phthalazinium, pyrazinium, pyrazolium, pyridazinium, pyridinium, pyrimidinium, quinazolinium, quinolinium, iso-quinolinium, quinoxalinium, tetrazolium, thiadiazolium, iso-thiadiazolium, thiazinium, thiazolium, iso-thiazolium, triazinium, triazolium, and iso-triazolium.

More preferably, [Cat⁺] has the formula:

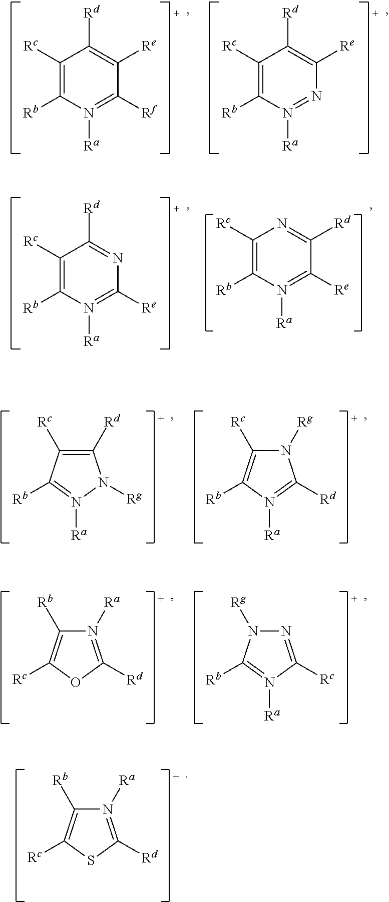

wherein: R^{a}, R^{b}, R^{c}, R^{d}, R^{e}, R^{f} and R^{g} are each independently selected from hydrogen, a C₁ to C₃₀, straight chain or branched alkyl group, a C₃ to C₈ cycloalkyl group, or a C₆ to C₁₀ aryl group, or any two of R^{b}, R^{c}, R^{d}, R^{e} and R^{f} attached to adjacent carbon atoms form a methylene chain —(CH₂)_{q}— wherein q is from 3 to 6; and wherein said alkyl, cycloalkyl or aryl groups or said methylene chain are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S) NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

R^{a} is preferably selected from C₁ to C₃₀, linear or branched, alkyl, more preferably C₂ to C₂₀ linear or branched alkyl, still more preferably, C₂ to C₁₀ linear or branched alkyl, and most preferably C₄ to C₈ linear or branched alkyl. Further examples include wherein R^{a} is selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl.

In the cations comprising an R^{g} group, R^{g} is preferably selected from C₁ to C₁₀ linear or branched alkyl, more preferably, C₁ to C₅ linear or branched alkyl, and most preferably R^{g} is a methyl group.

In the cations comprising both an R^{a} and an R^{g} group, R^{a} and R^{g} are each preferably independently selected from C₁ to C₃₀, linear or branched, alkyl, and one of R^{a} and R^{g} may also be hydrogen. More preferably, one of R^{a} and R^{g} may be selected from C₂ to C₂₀ linear or branched alkyl, still more preferably, C₂ to C₁₀ linear or branched alkyl, and most preferably C₄ to C₈ linear or branched alkyl, and the other one of R^{a} and R^{g} may be selected from C₁ to C₁₀ linear or branched alkyl, more preferably, C₁ to C₅ linear or branched alkyl, and most preferably a methyl group. In a further preferred embodiment, R^{a} and R^{g} may each be independently selected, where present, from C₁ to C₃₀ linear or branched alkyl and C₁ to C₁₅ alkoxyalkyl.

In further preferred embodiments, R^{b}, R^{c}, R^{d}, R^{e}, and R^{f} are independently selected from hydrogen and C₁ to C₅ linear or branched alkyl, and more preferably R^{b}, R^{c}, R^{d}, R^{e}, and R^{f} are hydrogen.

In this embodiment of the invention, [Cat⁺] preferably comprises a cation selected from:

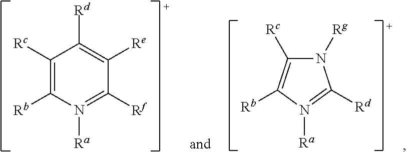

wherein: R^{a}, R^{b}, R^{c}, R^{d}, R^{e}, R^{f}, and R^{g} are as defined above.

More preferably, [Cat⁺] comprises a cation selected from:

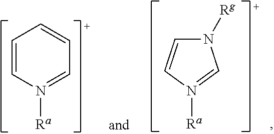

wherein: R^{a} and R^{g} are as defined above.

Also in accordance with this embodiment of the invention, [Cat⁺] may preferably comprise a cation selected from:

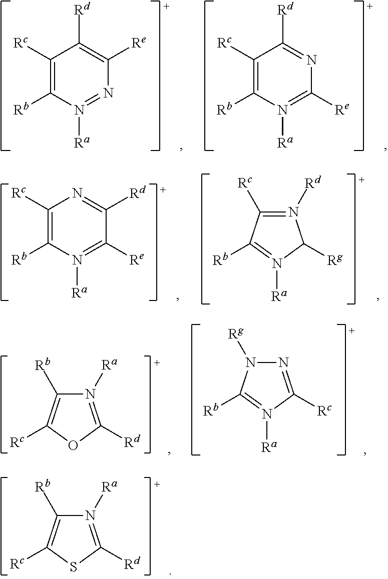

wherein: R^{a}, R^{b}, R^{c}, R^{d}, R^{e}, R^{f} and R^{g} are as defined above.

Specific examples of preferred nitrogen-containing aromatic heterocyclic cations that may be used according to the present invention include:

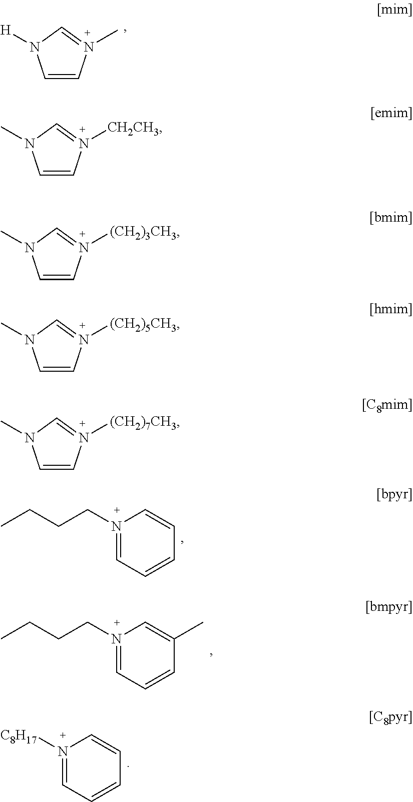

In another preferred embodiment of the invention, [Cat⁺] comprises a saturated heterocyclic cation selected from cyclic ammonium, 1,4-diazabicyclo[2.2.2]octanium, morpholinium, cyclic phosphonium, piperazinium, piperidinium, quinuclidinium, and cyclic sulfonium.

More preferably, [Cat⁺] comprises a saturated heterocyclic cation having the formula:

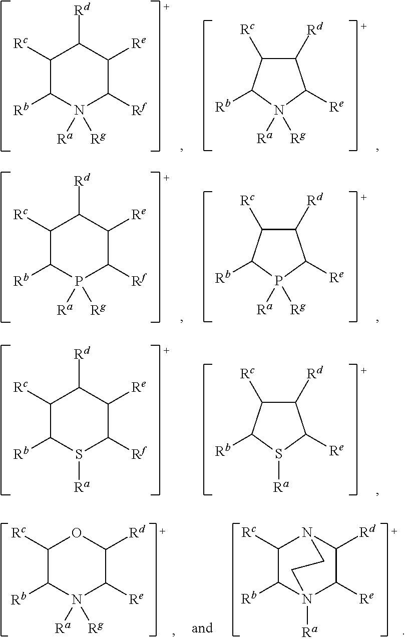

wherein: R^{a}, R^{b}, R^{c}, R^{d}, R^{e}, R^{f}, and R^{g} are as defined above.

Still more preferably, [Cat⁺] comprises a saturated heterocyclic cation having the formula:

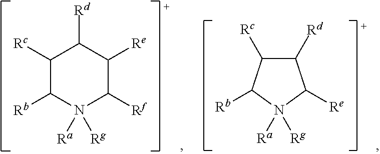

and is most preferably

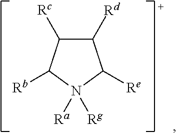

wherein: R^{a}, R^{b}, R^{c}, R^{d}, R^{e}, R^{f}, and R^{g} are as defined above.

A specific example of a preferred saturated heterocyclic cation suitable for use according to the present invention is 1-butyl-1-methylpyrrolidinium cation:

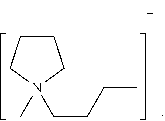

Also in accordance with this embodiment of the invention, [Cat⁺] may preferably comprise a saturated heterocyclic cation selected from:

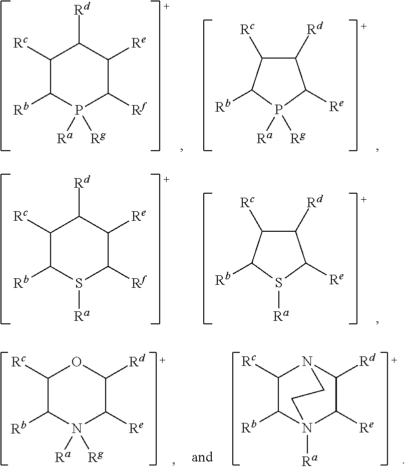

wherein: R^{a}, R^{b}, R^{c}, R^{d}, R^{e}, R^{f}, and R^{g} are as defined above.

In the saturated heterocyclic cations above, R^{a} is preferably selected from C₁ to C₃₀, linear or branched, alkyl, more preferably C₂ to C₂₀ linear or branched alkyl, still more preferably, C₂ to C₁₀ linear or branched alkyl, and most preferably C₄ to C₈ linear or branched alkyl. Further examples include wherein R^{a} is selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl.

In the saturated heterocyclic cations comprising an R^{g} group, R^{g} is preferably selected from C₁ to C₁₀ linear or branched alkyl, more preferably, C₁ to C₅ linear or branched alkyl, and most preferably R^{g} is a methyl group.

In the saturated heterocyclic cations comprising both an R^{a} and an R^{g} group, R^{a} and R^{g} are each preferably independently selected from C₁ to C₃₀, linear or branched, alkyl, and one of R^{a} and R^{g} may also be hydrogen. More preferably, one of R^{a} and R^{g} may be selected from C₂ to C₂₀ linear or branched alkyl, still more preferably, C₂ to C₁₀ linear or branched alkyl, and most preferably C₄ to C₈ linear or branched alkyl, and the other one of R^{a} and R^{g} may be selected from C₁ to C₁₀ linear or branched alkyl, more preferably, C₁ to C₅ linear or branched alkyl, and most preferably a methyl group. In a further preferred embodiment, R^{a} and R^{g} may each be independently selected, where present, from C₁ to C₃₀ linear or branched alkyl and C₁ to C₁₅ alkoxyalkyl.

In further preferred embodiments, R^{b}, R^{c}, R^{d}, R^{e}, and R^{f} are independently selected from hydrogen and C₁ to C₅ linear or branched alkyl, and more preferably R^{b}, R^{c}, R^{d}, R^{e}, and R^{f} are hydrogen.

In accordance with the present invention, [X⁻] may comprise one or more anions selected from hydroxides, halides, perhalides, pseudohalides, sulphates, sulphites, sulfonates, sulfonimides, phosphates, phosphites, phosphonates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, perchlorate, halometallates, amino acids and borates.

Thus, [X⁻] may represent one or more anions selected from:
a) a halide anion selected from: F⁻, Cl⁻, Br⁻, I⁻;
b) a perhalide anion selected from: [I₃]⁻, [I₂Br]⁻, [IBr₂]⁻, [Br₃]⁻, [Br₂C]⁻, [BrCl₂]⁻, [ICl₂]⁻, [I₂Cl]⁻, [Cl₃]⁻;
c) a pseudohalide anion selected from: [N₃]⁻, [NCS]⁻, [NCSe]⁻, [NCO]⁻, [CN]⁻;
d) a sulphate anion selected from: [HSO₄]⁻, [SO₄]²⁻, [R^{k}OSO₂O]⁻;
e) a sulphite anion selected from: [HSO₃]⁻, [SO₃]²⁻, [R^{k}OSO₂]⁻;
f) a sulfonate anion selected from: [R^{j}SO₂O]⁻;
g) a sulfonimide anion selected from: [(R^{j}SO₂)₂N]⁻;
h) a phosphate anion selected from: [H₂PO₄]⁻, [HPO₄]²⁻, [PO₄]³⁻, [R^{k}OPO₃]²⁻, [(R^{k}O)₂PO₂]⁻,
i) a phosphite anion selected from: [H₂PO₃]⁻, [HPO₃]²⁻, [R^{k}OPO₂]²⁻, [(R^{k}O)₂PO]⁻;
j) a phosphonate anion selected from: [R^{j}PO₃]²⁻, [R^{j}P(O)(OR^{k})O]⁻;
k) a methide anion selected from: [(R^{j}SO₂)₃C]⁻;
l) a borate anion selected from: [bisoxalatoborate], [bismalonatoborate];
m) a carboxylate anion selected from: [R^{k}CO₂]⁻;
n) an azolate anion selected from: [3,5-dinitro-1,2,4-triazolate], [4-nitro-1,2,3-triazolate], [2,4-dinitroimidazolate], [4,5-dinitroimidazolate], [4,5-dicyano-imidazolate], [4-nitroimidazolate], [tetrazolate];
o) a sulfur-containing anion selected from: thiocarbonates (e.g. [R^{k}OCS₂]⁻); thiocarbamates and (e.g. [R^{k}₂NCS₂]⁻); thiocarboxylates (e.g. [R^{j}CS₂]⁻); thiophosphates (e.g. [(R^{k}O)₂PS₂]⁻); thiosulfonates (e.g. [RS(O)₂S]⁻); and thiosulfates (e.g. [ROS(O)₂S]⁻); and
p) a nitrate ([NO₃]⁻) or nitrite ([NO₂]⁻) anion;
q) a carbonate anion selected from [CO₃]²⁻, [HCO₃]⁻, [R^{k}CO₃]⁻; preferably [MeCO₃]⁻;
wherein: R^{j} and R^{k} are independently selected from the group consisting of C₁-C₁₀ alkyl, C₆ aryl, C₁-C₁₀ alkyl(C₆)aryl, and C₆ aryl(C₁-C₁₀)alkyl each of which may be substituted by one or more groups selected from: fluoro, chloro, bromo, iodo, C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl, and wherein R^{j} may also be fluorine, chlorine, bromine or iodine.

In one preferred embodiment, [X⁻] comprises a halide or perhalide anion selected from: [F]⁻, [Cl]⁻, [Br]⁻, [I]⁻, [I₃]⁻, [I₂Br]⁻, [IBr₂]⁻, [Br₃]⁻, [Br₂Cl]⁻, [BrCl₂]⁻, [ICl₂]⁻, [I₂Cl]⁻, [Cl₃]⁻. More preferably [X⁻] comprises a halide or perhalide anion selected from: [F]⁻, [Cl]⁻, [Br]⁻, [I]⁻, [I₂Br]⁻, [IBr₂]⁻, [Br₂Cl]⁻, [BrCl₂]⁻, [ICl₂]⁻, [I₂Cl]⁻.

In a further preferred embodiment, [X⁻] comprises an oxygen-containing anion selected from: [NO₃]⁻, [NO₂]⁻, [H₂PO₄]⁻, [HPO₄]²⁻, [PO₄]³⁻, [R^{k}OPO₃]²⁻, [(R^{k}O)₂PO₂]⁻, [H₂PO₃]⁻, [HPO₃]²⁻, [R^{k}OPO₂]²⁻, [(R^{k}O)₂PO]⁻, [R^{j}PO₃]²⁻, [R^{j}P(O)(OR^{k})O]⁻, wherein R^{j} and R^{k} are as defined above. Further examples of anions in this category include: [MeOPO₃]²⁻, [EtOPO₃]²⁻, [(MeO)₂PO₂]⁻, [(EtO)₂PO₂]⁻, [MePO₃]²⁻, [EtPO₃]²⁻, [MeP(O)(OMe)O]⁻, [EtP(O)(OEt)O]⁻.

In a further preferred embodiment, [X⁻] comprises a carboxylate anion selected from [R^{k}CO₂]⁻; wherein R^{k} is as defined above. Further examples of anions in this category include: [HCO₂]⁻, [MeCO₂]⁻, [EtCO₂]⁻, [CH₂(OH)CO₂]⁻, [CH₃CH(OH)CH₂CO₂]⁻, [PhCO₂]⁻, salicylate, alaninate, argininate, asparaginate, aspartate, cysteinate, glutamate, glutaminate, glycinate, histidinate, isoleucinate, leucinate, lysinate, methioninate, phenylalaninate, prolinate, serinate, threoninate, tryptophanate, tyrosinate, valinate, N-methylglycinate, 2-aminobutyrate, 2-aminoisobutyrate, 2-amino-4-aminooxy-butyrate, 2-(methylguanidino)-ethanoate, 2-pyrrolidone-5-carboxylate, piperidine-2-carboxylate, and 1-piperidinepropionate.

In a further preferred embodiment, [X⁻] comprises an anion comprising an electron-rich sulfur or selenium moiety. Examples include: anions as defined above comprising pendant thiol, thioether, or disulfide substituents, [NCS]⁻, [NCSe]⁻, [R^{k}OCS₂]⁻, [R^{k}₂NCS₂]⁻, [R^{k}CS₂]⁻, [(R^{k}O)₂PS₂]⁻, [R^{j}S(O)₂S]⁻ and [R^{j}OS(O)₂S]⁻, wherein R^{j} and R^{k} are as defined above. Further examples of anions in this category include: [CH₂(SH)CO₂]⁻, [CH₃CH₂(SH)CO₂]⁻, [CH₃CS₂]⁻, [CH₃CH₂CS₂]⁻, [PhCS₂]⁻, [(MeO)₂PS₂]⁻, [(EtO)₂PS₂]⁻, [(PhO)₂PS₂]⁻, [(CH₃)₂NCS₂]⁻, [(CH₃CH₂)₂NCS₂]⁻, [Ph₂NCS₂]⁻, [CH₃OCS₂]⁻, [CH₃CH₂OCS₂]⁻, [PhOCS₂]⁻,

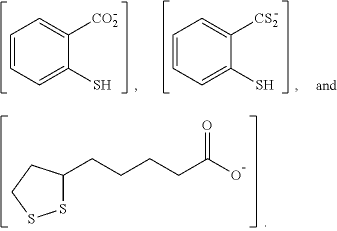

In a further preferred embodiment, [X⁻] comprises a sulfur-containing anion selected from sulphate anions ([HSO₄]⁻, [SO₄]²⁻, [R^{k}OSO₂O]⁻), sulphite anions ([HSO₃]⁻, [SO₃]²⁻, [R^{k}OSO₂]⁻) and sulfonate anions ([R^{j}SO₂O]⁻). Further examples of anions in this category include: [FSO₂O]⁻, [CF₃SO₂O]⁻, [MeSO₂O]⁻, [PhSO₂O]⁻, [4-MeCeH₄SO₂O]⁻, [dioctylsulfosuccinate]⁻, [MeOSO₂O]⁻, [EtOSO₂O]⁻, [C₈H₁₇OSO₂O]⁻, and [MeOSO₂]⁻, [PhOSO₂]⁻.

In a further preferred embodiment, [X⁻] comprises a carbonate anion selected from [R^{k}CO₃]⁻; wherein R^{k} is defined as above. Preferably, where [X⁻] comprises a carbonate anion selected from selected from [R^{k}CO₃]⁻, R^{k} is selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl. More preferably R^{k} is selected from methyl, ethyl, n-propyl, n-butyl, and most preferably R^{k} is methyl.

In a further preferred embodiment, [X⁻] may comprise an anion selected from [OH]⁻ and [SH]⁻.

In a particularly preferred embodiment of the invention, [X⁻] may comprise an anion selected from [CO₃]²⁻, [HCO₃]⁻, [MeCO₃]⁻, [OH]⁻, and [SH]⁻, most preferably an anion selected from [MeCO₃]⁻ and [OH]⁻.

In a further embodiment of the invention, [X⁻] may comprise a fluorinated anion selected from: [BF₄], [CF₃BF₃]⁻, [CF₃CF₂BF₃]⁻, [PF₆]⁻, [CF₃PF₅]⁻, [CF₃CF₂PF₅]⁻, [(CF₃CF₂)₂PF₄]⁻; and [(CF₃CF₂)₃PF₃]⁻. However, fluorinated anions of this type are generally less preferred in comparison with the anion types disclosed above.

The present invention is not limited to ionic liquids comprising anions and cations having only a single charge. Thus, the formula [Cat⁺][X⁻] is intended to encompass ionic liquids comprising, for example, doubly, triply and quadruply charged anions and/or cations. The relative stoichiometric amounts of [Cat⁺] and [X⁻] in the ionic liquid are therefore not fixed, but can be varied to take account of cations and anions with multiple charges. For example, the formula [Cat⁺][X^{]} should be understood to include ionic liquids having the formulae [Cat⁺]₂[X²⁻]; [Cat²⁺] [X⁻]₂; [Cat²⁺][X²⁻]; [Cat⁺]₃[X³⁻]; [Cat³⁺][X⁻]₃ and so on.

It will also be appreciated that the present invention is not limited to ionic liquids comprising a single cation and a single anion. Thus, [Cat⁺] may, in certain embodiments, represent two or more cations, such as a statistical mixture of 1,3-dimethylimidazolium, 1-ethyl-3-methylimidazolium and 1-3-diethylimidazolium. Similarly, [X⁻] may, in certain embodiments, represent two or more anions, such as a mixture of tribromide ([Br₃]⁻) and bistriflimide ([N(SO₂CF₃)₂]⁻).

In one embodiment, the ionic liquid used in the process of the present invention is tributylmethylammonium methylcarbonate.

In another embodiment, the ionic liquid used in the process of the present invention is 1-butyl-1-methylpyrrolidinium methylcarbonate.

In a further embodiment, the ionic liquid used in the process of the present invention is tetramethylammonium hydroxide.

Formation of alkanolamines of formula I and II in accordance with the present invention is believed to follow the following reaction pathway:

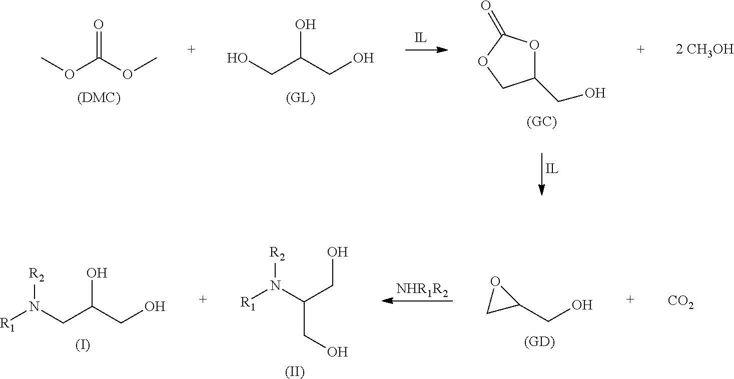

Dimethylcarbonate (DMC) and glycerol (GL) react in the presence of an ionic liquid catalyst to form a glycerol carbonate (GC) intermediate which is subsequently converted to glycidol (GD). Reaction of glycidol with an amine subsequently yields the desired alkanolamine.

It has been surprisingly found that selectivity for the production of the intermediate GD may be enhanced in an ionic liquid catalysed one-pot synthetic reaction of GL and DMC, whilst maintaining high conversion, by conducting the reaction at a temperature of from 100° C. to 160° C. and using a molar ratio of glycerol to dimethylcarbonate of from 1:4 to 1:10. It is particularly surprising that these reaction conditions lead to both high conversion and superior selectivity for the preparation of glycidol intermediate based on known prior art methods for the synthesis of GD from GL and DMC using an ionic liquid catalyst.

S. M. Gade et al., Catalysis Communications, 27, 2012, pages 184 to 188 (hereinafter referred to as “Gade et al”), reports a one-pot synthesis of GD from GL and DMC under mild conditions using an ionic liquid catalyst. However, selectivity was not affected significantly by changes in GL:DMC molar ratio according to that disclosure. According to those investigations in Gade et al, the highest GD selectivity observed was only 55%, for a GL:DMC molar ratio of 1:2. This reported selectivity is still very low. These results are represented graphically in FIG. 2; decreasing selectivity is observed when the GL:DMC ratio is changed from 2:1 to 3:1.

Gade et al also reports the effect of temperature on conversion and selectivity at three different temperatures (70, 80 and 90° C.). Conversion of GL was found to increase significantly with increase in temperature from 70 to 80° C. However, no further improvement in conversion was observed on increasing the reaction temperature from 80 to 90° C.

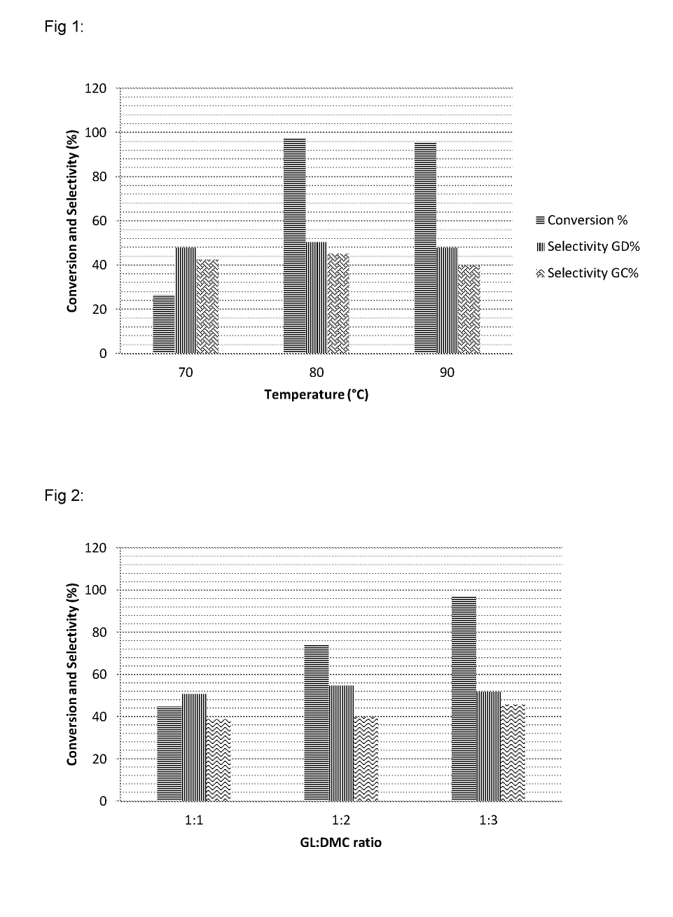

Further, and of note, changes in reaction temperature were not found to significantly affect GD selectivity, which is consistently shown to be around 50%, and actually decreasing with increasing temperature, when tested according to the standard operating conditions reported in Gade et al. These results are represented graphically in FIG. 1.

It is therefore entirely unexpected that the conditions for the reaction of GL and DMC according to step i) of the process of the invention would lead to high conversion as well as high selectivity for GD in the light of the information in Gade et al. High selectivity for the preparation of GD increases the yield of alkanolamine of formula I and/or II formed following the subsequent reaction of GD with an amine of formula III in step ii) of the process of the invention.

According to one embodiment of the invention, R₁ and R₂ of formula I, II or III are independently selected from hydrogen, a C₁ to C₈, straight chain or branched alkyl group, a C₂ to C₈ straight chain or branched alkenyl group, a C₃ to C₈ cycloalkyl group, a C₆ to C₁₀ aryl group, a 3 to 10 membered heterocyclic group, or R₁ and R₂ of formula I, II or III together with the nitrogen atom to which they are attached form a heterocyclic group, wherein said alkyl, alkenyl, cycloalkyl, aryl and heterocyclic groups are unsubstituted or may be substituted by one to three groups selected from: C₁ to C₆ alkoxy, C₂ to C₈ alkoxyalkoxy, C₃ to C₈ cycloalkyl, C₆ to C₁₀ aryl, 3 to 10 membered heterocyclic, C₇ to C₁₀ aralkyl, 3 to 10 membered heterocyclic-C₁ to C₄ alkyl, —OH, —CH₂CH(OH)CH₂(OH), —CH(CH₂OH)₂, or —NR₃R₄, wherein R₃ and R₄ are independently selected from hydrogen or C₁ to C₆ straight chain or branched alkyl group.

In another embodiment, R₁ of formula I, II or III is selected from a polysaccharide group, preferably derived from chitosan polysaccharide, or a polyethylene oxide group.

In a preferred embodiment of the invention, R₁ and R₂ of formula I, II or III are independently selected from hydrogen, a C₁ to C₆, straight chain or branched alkyl group, a C₃ to C₆ cycloalkyl group, a C₆ to C₁₀ aryl group, a 3 to 8 membered heterocyclic group, or R₁ and R₂ of formula I, II or III together with the nitrogen atom to which they are attached form a heterocyclic group, wherein said alkyl, cycloalkyl, aryl and heterocyclic groups are unsubstituted or may be substituted by one to three groups selected from: C₁ to C₆ alkoxy, C₂ to C₈ alkoxyalkoxy, C₃ to C₆ cycloalkyl, C₆ to C₁₀ aryl, 3 to 8 membered heterocyclic, C₇ to C₁₀ aralkyl, 3 to 10 membered heterocyclic-C₁ to C₄ alkyl, —OH, —CH₂CH(OH)CH₂(OH), —CH(CH₂OH)₂, or —NR₃R₄, wherein R₃ and R₄ are independently selected from hydrogen or C₁ to C₆ straight chain or branched alkyl group.

In a further preferred embodiment, R₁ and R₂ of formula I, II or III are independently selected from hydrogen, a C₁ to C₆, straight chain or branched alkyl group, a C₃ to C₆ cycloalkyl group, a C₆ to C₁₀ aryl group, a 3 to 8 membered heterocyclic group, or R₁ and R₂ of formula I, II or III together with the nitrogen atom to which they are attached form a heterocyclic group, wherein said alkyl, cycloalkyl, aryl, heterocylic groups are unsubstituted or may be substituted by one to three groups selected from: C₁ to C₆ alkoxy, C₂ to C₈ alkoxyalkoxy, C₃ to C₆ cycloalkyl, C₆ to C₁₀ aryl, 3 to 8 membered heterocyclic, —OH, —CH₂CH(OH)CH₂(OH), —CH(CH₂OH)₂, or —NR₃R₄, wherein R₃ and R₄ are independently selected from hydrogen or C₁ to C₆ straight chain or branched alkyl group.

In a more preferred embodiment of the invention, R₁ and R₂ of formula I, II or III are independently selected from hydrogen and a C₁ to C₆, straight chain or branched alkyl group, or R₁ and R₂ of formula I, II or III together with the nitrogen atom to which they are attached form a heterocyclic group, wherein said alkyl group or said heterocyclic group are unsubstituted or may be substituted by one to three groups selected from: C₁ to C₆ alkoxy, C₂ to C₈ alkoxyalkoxy, C₃ to C₆ cycloalkyl, C₆ to C₈ aryl, 3 to 8 membered heterocyclic, —OH, —CH₂CH(OH)CH₂(OH), —CH(CH₂OH)₂, or —NR₃R₄, wherein R₃ and R₄ are independently selected from hydrogen or C₁ to C₆ straight chain or branched alkyl group.

In a still more preferred embodiment, R₁ and R₂ of formula I, II or III are independently selected from hydrogen and a C₁ to C₆, straight chain or branched alkyl group wherein said alkyl group is unsubstituted or may be substituted by one to three groups selected from: C₃ to C₆ cycloalkyl, C₆ to C₈ aryl, —OH and —NR₃R₄, wherein R₃ and R₄ are independently selected from hydrogen or C₁ to C₆ straight chain or branched alkyl group.

In yet a still more preferred embodiment, R₁ and R₂ of formula I, II or III are independently selected from hydrogen and a C₁ to C₆, straight chain or branched alkyl group wherein said alkyl group is unsubstituted or may be substituted by one to three groups selected from: —OH and —NR₃R₄, wherein R₃ and R₄ are independently selected from hydrogen or C₁ to C₆ straight chain or branched alkyl group.

Examples of straight chain and branched alkyl groups in accordance with the present invention include methyl, ethyl, n-propyl, isopropyl, n-butyl, isobutyl, sec-butyl, t-butyl, n-pentyl, n-hexyl, isohexyl, n-heptyl and n-octyl. Preferred alkyl groups include methyl, ethyl, n-propyl, isopropyl, n-butyl, isobutyl and t-butyl.

Particularly preferred alkanolamines of formula I and II preparable by the process of the present invention are those wherein:
1) R₁ is H and R₂ is H;
2) R₁ is H and R₂ is methyl;
3) R₁ is H and R₂ is ethyl;
4) R₁ is H and R₂ is n-propyl;
5) R₁ is H and R₂ is isopropyl
6) R₁ is H and R₂ is n-butyl;
7) R₁ is H and R₂ is t-butyl;
8) R₁ is methyl and R₂ is methyl;
9) R₁ is ethyl and R₂ is ethyl;
10) R₁ is n-propyl and R₂ is n-propyl;
11) R₁ is isopropyl and R₂ is isopropyl;
12) R₁ is n-butyl and R₂ is n-butyl;
13) R₁ is t-butyl and R₂ is t-butyl;
14) R₁ and R₂ are as defined in embodiments 1) to 12) above and R₂ is also substituted with an —OH group;
15) R₁ and R₂ is as defined in embodiments 1) to 13) above and R₂ is also substituted with —NR₃R₄, wherein R₃ and R₄ are independently selected from hydrogen or C₁ to C₆ straight chain or branched alkyl group; and
16) R₁ is selected from —CH₂CH(OH)CH₂(OH) or —CH(CH₂OH)₂ and R₂ is selected from methyl, ethyl, n-propyl, n-butyl or t-butyl.

In accordance with the present invention, in step i) of the process the molar ratio of glycerol to dimethylcarbonate is from 1:4 to 1:10, preferably from 1:5 to 1:8, such as for example 1:6 to 1:7. Thus, exemplary molar ratios of glycerol to dimethylcarbonate include: 1:5, 1:6, 1:7 or 1:8.

Step i) of the process is conducted at a temperature of 100° C. to 160° C., preferably the process is conducted at a temperature of 110° C. to 140° C., more preferably from 115° C. to 130° C. A temperature of from 115° C. to 125° C., for example 120° C., has been found to be particularly beneficial with step i) of the process of the present invention.

Heating in step i) of the process may be accomplished using any suitable method, of which those skilled in the art would be readily aware. For example, the reaction in step i) may be heated using conventional thermal methods, microwave heating or employing other heat sources such as ultrasound or infrared radiation. In one embodiment of the invention, heating in step i) of the process is accomplished by conventional thermal heating. In another embodiment of the invention, heating in step i) of the process is accomplished by microwave heating in a microwave reactor. Ionic liquids may be readily integrated into a microwave reaction due to their high microwave absorption capabilities and therefore can support a fast and clean process.

Where a microwave reactor apparatus is used with the process of the present invention, heating is provided by microwave energy (i.e. electromagnetic radiation of a frequency of about 10⁸ Hz to 10¹² Hz) generated by a magnetron, typically operating at a frequency of 2450 MHz. The reaction mixture may be heated in open or, preferably, sealed vessels. Preferably, the microwave reactor is automated such that a particular temperature, maximum pressure, maximum power output and hold time can be specified during operation. Suitable microwave reactors for use with the present invention include the CEM Explorer and Anton Paar Monowave 300 microwave reactors.

Step i) of the process may be conducted at a pressure of from 10,000 to 1,500,000 Pa (0.1 to 15 bar), more preferably from 10,000 to 1,000,000 Pa (0.1 to 10 bar), and most preferably 50,000 to 500,000 Pa (0.5 to 5 bar).

As would be understood by those of skill in the art, the ionic liquid and the glycerol and dimethyl carbonate reactants may be reacted in step i) by means of continuous processes or batch processes. Any conventional liquid-liquid or gas-liquid contactor apparatus may be used in accordance with the present invention. For instance, the ionic liquid and the glycerol and dimethyl carbonate reactants may be reacted using a counter-current liquid-liquid contactor, a co-current liquid-liquid contactor, a counter-current gas-liquid contactor, a co-current gas-liquid contactor, a liquid-liquid batch contactor, or a gas-liquid batch contactor.

If desired, step i) of the process may be conducted in the presence of a solvent which is compatible with the ionic liquid, glycerol, dimethyl ether, glycerol carbonate and glycidol. The use of a solvent may be appropriate where it is desired to modify the viscosity of an ionic liquid. Suitable solvents for this purpose are non-basic aprotic polar solvents, such as ethyl acetate, acetone, diethyl ether, acetonitrile, dimethylsulfoxide, dimethylformamide and sulfolane (tetrahydrothiophene 1,1-dioxide). The presence of a solvent may also facilitate subsequent separation of the glycidol product from the ionic liquid. Thus, in an embodiment of the invention, solvent is present in an amount up to 80 wt % based on the total weight of the reaction mixture in step i) of the process. For example, solvent may be present in an amount of from 40 wt % to 80 wt %; from 50 wt % to 80 wt %; or from 60 wt % to 80 wt %, based on the total weight of the reaction mixture in step i) of the process.

Alternatively, solvent is present in an amount less than 20 wt % based on the total weight of the reaction mixture in step i) of the process. In another embodiment of the invention, solvent is present in an amount less than 10 wt %, based on the total weight of the reaction mixture in step i) of the process. In a further embodiment, the reaction is conducted substantially in the absence of a solvent (i.e. less than 10 wt %, preferably less than 5 wt %, for example 2 wt %, 1 wt % or 0 wt %) in step i) of the process.

The ionic liquid may be supported on a solid, preferably porous, carrier material which is compatible the process of the present invention. Suitable solid carriers for use in this embodiment of the invention include silica alumina, silica-alumina, and activated carbon. In general, supported ionic liquids for use according to this embodiment of the invention comprise from 50% by weight to 1% by weight of ionic liquid, and more preferably 20% by weight to 1% by weight of ionic liquid.

The amount of ionic liquid catalyst used in step i) of the process of the invention is not particularly limited and the skilled person is able to readily identify a suitable amount based on the amount of the reactants. For instance, the ionic liquid catalyst may be present in an amount corresponding to at least 2 mol % based on glycerol. However, it has been surprisingly found that an amount of at least 3 mol % ionic liquid catalyst based on glycerol is particularly beneficial for step i) of the process of the present invention. Thus, the amount of ionic liquid catalyst preferably used in step i) of the process is at least 3 mol %, more preferably at least 5 mol %, even more preferably at least 8 mol %, for example 10 mol %, based on glycerol.

In some embodiments of the invention, the ionic liquid is recycled after use in step i) of the process. Separation of the ionic liquid from product/by-product materials can readily be undertaken by a skilled person using known separation techniques, such as partitioning between different liquid phases (e.g. aqueous and organic liquid phases). Alternatively, advantage may be taken of the negligible vapour pressure of ionic liquids by separation of product/by-product materials into a vapour phase.

Step i) of the process is conducted over a suitable timescale to obtain quantitative or near quantitative (e.g. greater than 95%) conversion of glycerol. It will be appreciated that the rate of reaction will vary according to the ionic liquid that is used. In addition, other reaction parameters such as temperature, pressure and the choice of solvent, if any, may also influence the reaction rate. Quantitative or near quantitative conversion of glycerol is preferably obtained following a reaction time of up to 90 minutes, more preferably up to 60 minutes, still more preferably up to 30 minutes and most preferably up to 15 minutes.

Where heating of the reaction is accomplished in a microwave, quantitative or near quantitative conversion of glycerol is preferably obtained following a microwave reaction hold time of up to 90 minutes, more preferably up to 60 minutes, still more preferably up to 30 minutes and most preferably up to 15 minutes. Reference to “hold time” herein means the time a reaction mixture is held in a microwave reactor at a predetermined temperature, and not the total irradiation time of the reaction mixture.

In accordance with step ii) of the process of the invention, an alkanolamine compound of formula I and/or II, or a salt thereof, is prepared by reaction of glycidol with an amine group of formula III. Methods for producing alkanolamines by ring opening of epoxides are well known to a person of skill in the art. Such reactions are, for instance, described in U.S. Pat. No. 4,863,563, U.S. Pat. No. 2,089,569, EP 2295438 and Org. Lett., 2005, 7, pages 3649 to 3651.

In accordance with the present invention, step ii) of the process may be conducted by reacting the glycidol (GD) product of step i) with the amine of formula III, without first separating GD from the reaction mixture containing ionic liquid catalyst. Alternatively, GD may be separated from the reaction mixture before it is subsequently reacted with the amine of formula III. In either case, it is preferred that when GD is reacted with the amine of formula III in step ii) of the process, GD is added portion-wise, for example drop-wise, to the amine. This approach has been found to reduce the possibility of undesired side reactions, such as base catalysed polymerisation of GD.

Where glycidol is isolated prior to reaction with the amine of formula III in step ii), separation may by any suitable means of which the skilled person is aware. For instance, in one embodiment, glycidol is recovered from the reaction mixture using liquid-liquid extraction. Glycidol may be preferentially partitioned into an organic phase such as, for example, ethyl acetate, which may then be separated, for instance by gravity separation, for example, in a settling unit. Different phases may also be separated using, for example, a decanter, a hydrocyclone, electrostatic coalescer or a centrifuge.

Another suitable method for separation of glycidol from the reaction mixture is by azeotropic distillation, for instance, using cumene, as described, for example, in U.S. Pat. No. 3,374,153. Thus, a typical azeotropic distillation following that method comprises first removing any excess dimethyl carbonate and produced methanol under vacuum at room temperature. A large excess of cumene is then added, for example 50 equivalents of cumene per equivalent of glycidol, before the mixture is distilled under reduced pressure. In the method described in U.S. Pat. No. 3,374,153, glycidol is subsequently separated from cumene by extraction into water, before it is subjected to further fractional distillation. This final step is, however, less preferred in the context of the present invention, since glycidol also forms an azeotrope with water, and hydrolysis of glycidol during such a step may reduce the overall yield of glycidol.

Advantageously, it has been found that cumene is a suitable solvent for step ii) of the process of the invention. Therefore, it is preferred that glycidol is not separated from cumene prior to reaction with the amine of formula III in step ii) of the process of the invention. In this way, the alkanolamine formed, which is of much higher boiling point than cumene, can be easily separated from the solvent simply by removing cumene under a vacuum. Thus, in an embodiment of the invention, glycidol is separated from the reaction mixture before being reacted in step ii) of the process by azeotropic distillation, preferably using cumene. More preferably, the glycidol/cumene mixture obtained from azeoptropic distillation using cumene is used directly for reaction with the amine of formula III in step ii) of the process.

Step ii) of the process is conducted at a suitable temperature which leads to acceptable reaction rate, without leading to thermal decomposition of any of the reactants. Preferably, step ii) of the process is conducted at a temperature of 10° C. to 100° C., more preferably at a temperature of 30° C. to 70° C., still more preferably from 40° C. to 60° C., for example 50° C. As will be appreciated by the skilled person, where the amine of formula III is ammonia, higher temperatures may be required in order to obtain an acceptable reaction rate, and thereby compensate for the lower nucleophilicity associated with the nitrogen atom of ammonia. Thus, the temperature of step ii) may, for instance, be as high as 225° C. in the case where the amine of formula III is ammonia. Preferably, the temperature of the process of step ii) is 200° C. or less in the case where the amine of formula III is ammonia, more preferably the temperature of the process of step ii) is from 150 to 200° C. in the case where the amine of formula III is ammonia.

Heating in step ii) of the process may be accomplished using any suitable method, of which those skilled in the art would be readily aware, and may suitably include conventional thermal or microwave methods described hereinbefore.

Step ii) of the process may be conducted at a pressure of from 10,000 to 1,500,000 Pa (0.1 to 15 bar), more preferably from 10,000 to 1,000,000 Pa (0.1 to 10 bar), and most preferably 50,000 to 500,000 Pa (0.5 to 5 bar).

As would be understood by those of skill in the art, glycidol and the amine of formula III may be reacted in step ii) by means of continuous processes or batch processes. Any conventional liquid-liquid or gas-liquid contactor apparatus may be used in accordance with the present invention. For instance, glycidol and the amine of formula III may be reacted using a counter-current liquid-liquid contactor, a co-current liquid-liquid contactor, a counter-current gas-liquid contactor, a co-current gas-liquid contactor, a liquid-liquid batch contactor, or a gas-liquid batch contactor.

If desired, step ii) of the process may be conducted in the presence of a solvent which is compatible with the reactants. Suitable solvents for this purpose are non-basic aprotic polar and non-polar solvents, such as ethyl acetate, acetone, diethyl ether, acetonitrile, dimethylsulfoxide, dimethylformamide, sulfolane (tetrahydrothiophene 1,1-dioxide), dichloromethane, chloroform and cumene. In one embodiment of the invention, solvent is present in an amount less than 80 wt % based on the total weight of the reaction mixture in step ii) of the process. In another embodiment of the invention, solvent is present in an amount less than 50 wt % based on the total weight of the reaction mixture in step ii) of the process.

In a preferred embodiment of the invention, solvent is present in an amount less than 30 wt % based on the total weight of the reaction mixture in step ii) of the process. In a more preferred embodiment of the invention, solvent is present in an amount less than 10 wt %, based on the total weight of the reaction mixture in step ii) of the process. In a further embodiment, the reaction is conducted substantially in the absence of a solvent (i.e. less than 10 wt %, preferably less than 5 wt %, for example 2 wt %, 1 wt % or 0 wt %) in step ii) of the process.

As will be appreciated by the skilled person, where the amine of formula III is a primary amine, in other words where one of R₁ and R₂ is H, or where the amine of formula III is ammonia, it is possible for the reaction of a single amine compound with multiple glycidol molecules leading to the corresponding dimer or trimer alkanolamine compounds being formed. The skilled person will be aware that multiple ring-opening reactions with a single amine molecule may be substantially eliminated by using a large excess of the amine compound of formula III in comparison with glycidol. A similar effect may also be achieved by adding glycidol portionwise to the amine; representing a further advantage of that approach to mixing of glycidol and the amine of formula III.

The amount of glycidol used in step ii) of the process of the invention is therefore not particularly limited and the skilled person is able to readily identify a suitable amount based on the amount of the amine of formula III to be reacted. As mentioned above, where the amine of formula III is a primary amine or ammonia, a large excess of amine (e.g. 5 to 10 equivalents of the amine) may be used to reduce the possibility of a single amine compound reacting with multiple glycidol molecules. Thus, in a preferred embodiment, where the amine of formula III is a primary amine or ammonia, at least 5 equivalents of the amine are used for the reaction in step ii). More preferably at least 10 equivalents are used. However, a lower excess of amine, for example between 1.1 and 1.3 equivalents, has also been found to be suitable, particularly with dropwise addition of the glycidol to the amine, where the amine is a secondary amine.

The alkanolamines obtained in step ii) may be purified or used directly as part of a crude product mixture. Any suitable method of purification of which the skilled person is aware may be used, as desired. For instance, alkanolamines of formula I and/or II may be isolated from a crude product mixture by means of distillation, for example at reduced pressure. It has been found that when alkanolamines of formula I and/or II obtained by the process of the invention are used as acid-gas sweeteners, improved results are obtained when a purified sample of alkanolamine is used rather than a crude product mixture. Thus, it is preferred that alkanolamines of formula I and/or II are separated from the crude product mixture, for example by distillation, before being used as acid-gas sweeteners.

The reaction of glycidol with an amine of formula III typically leads to the formation of a mixture of structural isomers corresponding to compounds of formula I and II herein; the isomer according to formula I typically being the major component. Since the different geometrical isomers corresponding to alkanolamine compounds of formula I and II herein are both suitable for use as acid-gas sweeteners, it is not necessary to separate these isomers prior to their use in acid-gas absorption. However, if it is desired, the structural isomers may be separated by known separation methods, such as chromatographic methods.

It has been found that alkanolamines of formula I and II are particularly advantageous when used as acid-gas absorbents, having absorption rates which are comparable with those of known alkanolamine absorbents, such as MEA and DEA. The characteristic diol functionalities of the alkanolamines according to formula I and formula II are thought to confer desirable properties, such as high absorption rate and relatively high boiling point (above about 200° C.), making alkanolamines of formula I and II particularly useful as acid-gas absorbents.

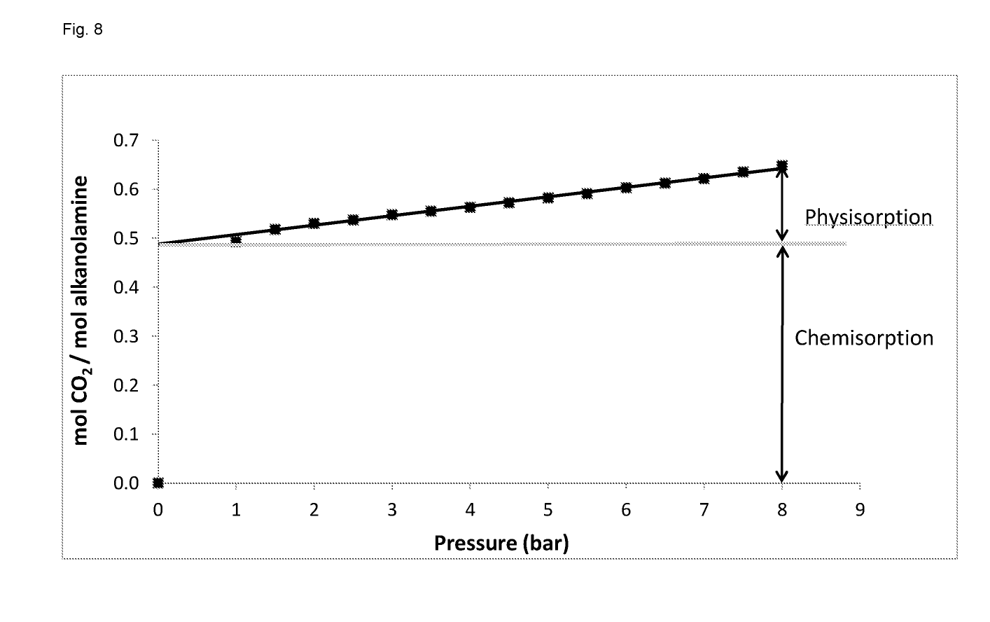

Without being bound by any particular theory, it is believed that absorption of CO₂ and/or H₂S by the alkanolamine compounds of formula I and/or II occurs both through chemical absorption, as discussed hereinbefore, as well as physical absorption processes. This is illustrated experimentally by absorption of CO₂ exceeding the theoretical maximum of 0.5 mol CO₂ per mol of alkanolamine when CO₂ partial pressure is increased (see FIG. 8).

Thus, in another embodiment, the process of the invention further comprises the steps of:
iii) contacting a CO₂ and/or H₂S containing gaseous stream with an alkanolamine compound of formula I and/or an alkanolamine compound of formula II, or salts thereof, prepared in step ii); and
iv) obtaining a treated gaseous stream having a reduced content of CO₂ and/or H₂S compared with the CO₂ and/or H₂S containing gaseous stream of step iii).

It will be appreciated that contacting step iii) of the process requires the presence of at least one alkanolamine compound according to formula I or formula II. Thus, in one embodiment of the invention one compound of formula I is used for contacting step iii) of the process. In another embodiment, one compound of formula II is used for contacting step iii) of the process. Mixtures of alkanolamine compounds may also be used for contacting step iii) of the process. Thus, a mixture of two or more alkanolamine compounds of formula I may be used for contacting step iii) of the process. Equally, a mixture of two or more alkanolamine compounds of formula II may be used for contacting step iii) of the process. Furthermore, a mixture of at least one compound of formula I and at least one compound of formula II may also be used for contacting step iii) of the process.

Preferably, the alkanolamine of formula I and/or II is used in contacting step iii) in the form of a solution. The total amount of alkanolamine compound according to formula I or II in the solution is preferably from 20% to 70% by weight, more preferably from 30% to 60% by weight, most preferably 30% to 50% by weight, for example 40% by weight.

Suitable solvents for use in contacting step iii) in the process of the invention include polar protic solvents including water, alcohols, for example methanol, ethanol, n-butanol and isopropanol. Preferably, the solvent is water. Thus, in an embodiment of the invention, the compound of formula I and/or formula II is provided in the form of an aqueous solution for use in contacting step iii).

Alternatively, the alkanolamine utilised in contacting step (iii) may be provided in a supported form. Suitable supports for use in the present invention include membrane supports. Examples of suitable polymeric membrane supports, including a polyethersulfone (PES) support, are included in the review of amine polymeric membranes (APM) by Nasir et al., World Academy of Science, Engineering and Technology 81, 2013, pp 465 to 468.

In one embodiment, the alkanolamine compound of formula I and/or formula II is used in contacting step iii) in supported form, wherein R₁ and R₂ of formula I and/or II do not form a heterocyclic group, R₁ is selected from a polysaccharide group, preferably a chitosan, or a polyethylene oxide group.

Although it is not necessary for achieving sufficient absorption of acid-gases with the alkanolamine of formula I and/or II, contacting step iii) may be performed in the presence of an activator compound which may enhance aborption characteristics of the alkanolamine of formula I and/or II. A suitable activator compound is, for example, piperazine.

Contacting step iii) may be performed in an absorber or contact column, preferably at a temperature of from 10 to 80° C., more preferably from 20 to 60° C. and most preferably from 30 to 50° C. For example, the gaseous stream may be contacted with the alkanolamine of formula I or II at a temperature at or around 30° C. The gaseous stream is preferably contacted with the alkanolamine of formula I or II at a pressure of from 100 to 2000 kPa, and more preferably from 200 to 1000 kPa. For example, the gaseous stream may be contacted with the carbon dioxide absorbent at a pressure at or around 500 kPa.

The removal of CO₂ and/or H₂S from a gaseous stream with an alkanolamine of formula I or II may form part of a batch or continuous process. In some embodiments, the alkanolamine is provided in the form of a solution where, following contact with the gaseous stream, the solution is sent to a desorber, or stripping column. The acid-gases may be separated from the solution in a desorber by reducing the partial pressure of the acid-gas over the solution and/or stripping the solution with steam. In some embodiments, steam stripping vapour may be produced where the alkanolamine is provided in the form of an aqueous solution by simply heating in a reboiler. A lean alkanolamine solution may then be recycled back to the absorber.

Contacting step iii) may be used to remove CO₂ and/or H₂S, and optionally one or more additional substances, from a number of different types of gaseous streams. For example, alkanolamines of formula I and/or II may be used to remove CO₂ and/or H₂S from the exhaust gas from a combustion process, such as the flue gases from furnaces and power plants. Alkanolamines of formula I and/or II may also used to remove CO₂ and/or H₂S, and optionally one or more additional substances, from hydrocarbon-containing gaseous streams, in particular methane-containing gaseous streams. Thus, alkanolamines of formula I and/or II may advantageously be used for the removal of CO₂ and/or H₂S, and optionally one or more additional substances, from natural gas and/or biogas. The alkanolamines of formula I and/or II may advantageously be used for the removal of CO₂ and/or H₂S from breathing gas mixtures in life support systems.

It will be appreciated that use of alkanolamines of formula I and/or II as acid-gas absorbents may be integrated into processing plants as one stage of a multi-stage processing of gaseous streams. For instance, alkanolamines of formula I and/or II produced by the process of the invention could be used in a natural gas refinery as one stage in the production of a commercial natural gas product, wherein other stages could include removal of nitrogen and removal of heavy hydrocarbons. Alternatively, alkanolamines of formula I and/or II produced by the process of the invention could be used in a flue gas treatment plant as one stage of a multi-stage processing of flue gases, where other stages could for instance include removal of particulates and catalytic conversion of NOx.

In another aspect, the present invention also provides the use of an alkanolamine compound of formula I and/or formula II prepared by a process described herein for removing CO₂ and/or H₂S from a CO₂ and/or H₂S containing gaseous stream. Thus, in one embodiment an alkanolamine compound of formula I and/or formula II prepared in accordance with the process of the invention is used for removing CO₂ and/or H₂S, and optionally one or more additional substances, from methane containing gaseous streams, such as natural gas, and/or biogas. In another embodiment the alkanolamine compound of formula I and/or formula II prepared in accordance with the process of the invention is used for removal of CO₂ and/or H₂S from a flue gas stream. In yet a still further embodiment the alkanolamine compound of formula I and/or formula II prepared in accordance with the process of the invention is used for removal of CO₂ and/or H₂S from breathing gas mixtures in life support systems.

The present invention will now be illustrated by way of the following examples and with reference to the following figures:

FIG. 1: Graphical representation of effect of temperature on conversion and selectivity as reported in Gade et al;

FIG. 2: Graphical representation of effect of GL:DMC ratio on conversion and selectivity as reported in Gade et al;

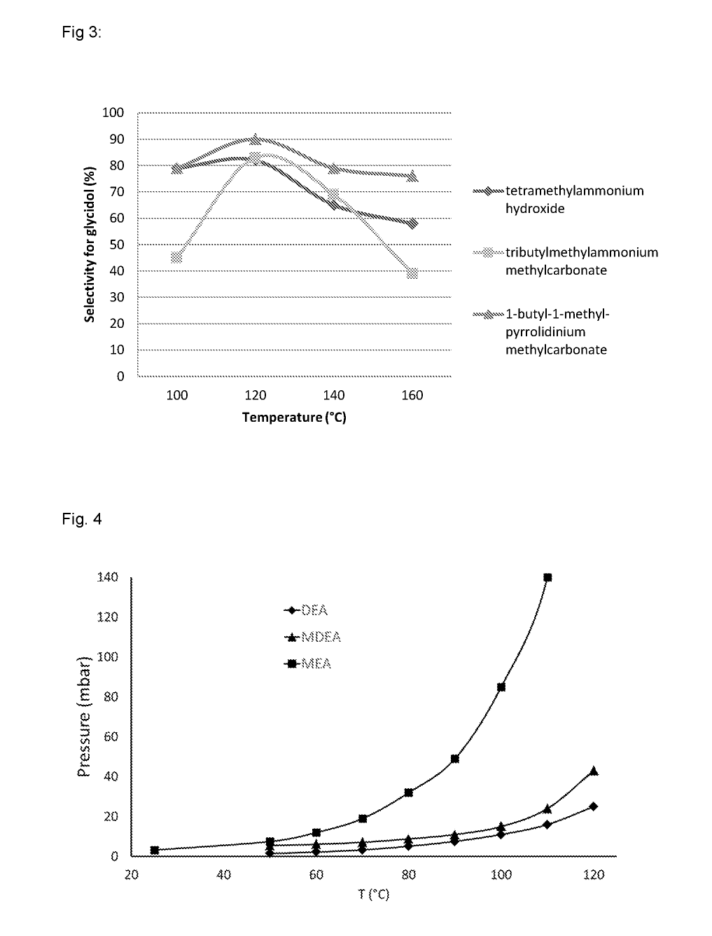

FIG. 3: Graphical representation of the effect of temperature (microwave heating with 15 minutes hold time) on glycidol selectivity for a glycidol synthesis wherein GL:DMC ratio is 1:5 using tributylmethylammonium methylcarbonate, 1-butyl-1-methylpyrrolidium methylcarbonate or tetramethylammonium hydroxide as ionic liquid catalyst;

FIG. 4: Graphical representation showing the vapour pressure-temperature profiles for industry standards MEA, DEA and MDEA;

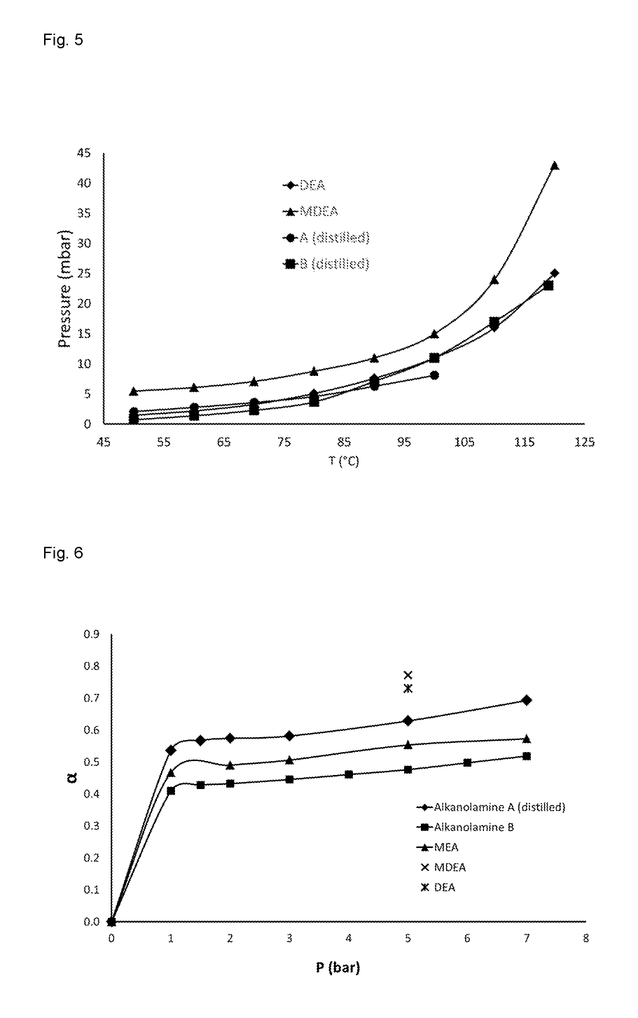

FIG. 5: Graphical representation showing the vapour pressure-temperature profiles for industry standards DEA and MDEA against alkanolamines A and B prepared in accordance with the process of the present invention;

FIG. 6: Graphical representation comparing the CO₂ absorption capacities of alkanolamines A and B prepared in accordance with the process of the invention and industry standards MEA, DEA and MDEA;

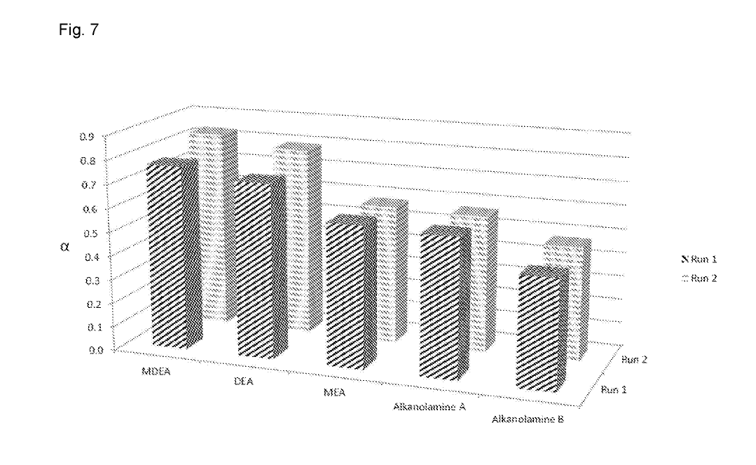

FIG. 7: Graphical representation illustrating reproducibility of results for repeated CO₂ uptake experiments for alkanolamines A and B prepared in accordance with the process of the invention, as well as for industry standards MEA, DEA and MDEA; and

FIG. 8: Graphical representation illustrating how an alkanolamine prepared in accordance with the process of the present invention exhibits both chemical and physical absorption characteristics.

### EXAMPLES

Preparation of Ionic Liquids

Tetramethylammonium hydroxide was prepared from a commercially available 25% solution of aqueous tetramethylammonium solution. Water was removed from the solution using a rotary evaporator.

Tributylmethylammonium methylcarbonate and 1-butyl-1-methylpyrrolidinium methylcarbonate were prepared according to the microwave-assisted synthesis of methylcarbonate salts reported in Holbrey et al., Green Chem., 2010, 12, pp 407 to 413.

Tributylamine (1.854 g, 10 mmol), DMC (0.90 g, 10 mmol) and methanol (2 ml) were added to 10 ml glass microwave process vial together with a magnetic stirring bar before the vial was sealed and placed inside a CEM Explorer microwave reactor. The solution was heated at 160° C. for 1 hour hold time with magnetic stirring. Tributylmethylammonium methylcarbonate was isolated after removal of the volatile solvent and excess DMC under reduced pressure.

1-butylpyrrolidine (1.272 g, 10 mmol), DMC (0.90 g, 10 mmol) and methanol (2 ml) were added to 10 ml glass microwave process vial together with a magnetic stirring bar before the vial was sealed and placed inside a CEM Explorer microwave reactor. The solution was heated at 140° C. for 1 hour hold time with magnetic stirring. 1-butyl-1-methylpyrrolidinium methylcarbonate was isolated after removal of the volatile solvent and excess DMC under reduced pressure.

Microwave Reactions

A CEM Explorer microwave reactor was used for performing microwave reactions, operating at a frequency of 2450 MHz with a maximum power output of 80 W. The ingredients were added to a 10 ml glass microwave process vial together with a magnetic stirrer bar before the vial was sealed and placed inside the reactor. Samples were then run for a predetermined time at a specified hold temperature. Run times referred to below, unless otherwise indicated, refer to the time a sample is held at a particular temperature, and not the total irradiation time.

Analysis of Product Samples

Following the reaction, samples were analysed by gas chromatography (GC) using an Agilent 6890N gas chromatograph with a HP-Innowax capillary column employing a He carrier gas operated according to the following: i) flow rate of 0.7 cm³ min⁻¹ at 50° C. for one minute; ii) linear gradient of 25° C. min⁻¹ to 200° C.; iii) linear gradient of 3° C. min⁻¹ from 200° C. 10 to 230° C.; and iv) 18 minutes hold at 230° C.

One-Pot Preparation of Glycidol from Dimethyl Carbonate and Glycerol

### Example 1

1-butyl-1-methylpyrrolidinium methylcarbonate (0.02173 g, 0.1 mmol) was combined with glycerol (0.093 g, 1 mmol) and dimethylcarbonate (0.45 g, 5 mmol) in a 20 ml sealed glass tube with a pressure rating of 1000 kPa (10 bar), along with a magnetic stirrer bar. The sealed glass tube was placed in an oil bath pre-heated to 120° C. and stirred for 15 minutes with vigorous magnetic stirring. The glass tube was then removed from the oil bath and allowed to cool to room temperature before a sample extracted for gas chromatography (GC) analysis.

### Example 2

The process of Example 1 was repeated, except that the reaction was heated for 30 minutes at 120° C. Catalyst loading was kept constant at 10 mol % based on glycerol and the same molar ratio of glycerol:dimethyl carbonate was employed (1:5).

### Example 3

1-butyl-1-methylpyrrolidinium methylcarbonate (0.02173 g, 0.1 mmol) was combined with glycerol (0.093 g, 1 mmol) and dimethylcarbonate (0.45 g, 5 mmol) in a 10 ml glass microwave process vial, along with a magnetic stirrer bar, before the vial was sealed. The sample was placed inside a CEM Explorer microwave reactor heated with magnetic stirring for a hold time of 15 minutes at 120° C. and a pressure of 550 kPa (5.5 bar), before the reaction mixture was analysed directly by gas chromatography (GC).

### Example 4

The process of Example 3 was repeated, except that tributylmethylammonium methyl carbonate was used in place of 1-butyl-1-methylpyrrolidinium methylcarbonate. Catalyst loading was kept constant at 10 mol % based on glycerol and the same molar ratio of glycerol:dimethyl carbonate was employed (1:5).

### Example 5

The process of Example 3 was repeated, except that tetramethylammonium hydroxide was used in place of 1-butyl-1-methylpyrrolidinium methylcarbonate. Catalyst loading was kept constant at 10 mol % based on glycerol and the same molar ratio of glycerol:dimethyl carbonate was employed (1:5).

### Example 6

The process of Example 3 was repeated, except that a molar ratio of glycerol:dimethyl carbonate of 1:8 was used. Catalyst loading was kept constant at 10 mol % based on glycerol.

### Example 7

The processes of Examples 3 to 5 were repeated for a range of different hold temperatures (100° C., 140° C. and 160° C.). Catalyst loading was kept constant at 10 mol % based on glycerol and the same molar ratio of glycerol:dimethyl carbonate was employed (1:5) in each case.

### Example 8

The process of Example 3 was repeated, except that a molar ratio of glycerol:dimethyl carbonate of 1:15 was used. Catalyst loading was kept constant at 10 mol % based on glycerol.

Table 1 below shows the results of Examples 1 to 8. The results in Table 1 (corresponding to Entries 3, 4 and 7 to 16) have also been used for generating a graphical representation (FIG. 3).

TABLE 1

| | | | | | GD | GC |
|---|---|---|---|---|---|---|
| | | Temp. | GL:DMC | Conversion | selectivity | selectivity |
| Entry | Catalyst | (° C.) | ratio | of GL (%) | (%) | (%) |
| 1¹ | 1-butyl-1- | 120° C. | 1:5 | 100 | 85 | 15 |
| | methylpyrrolidinium | | | | | |
| | methylcarbonate | | | | | |
| 2² | 1-butyl-1- | 120° C. | 1:5 | 100 | 86 | 12 |
| | methylpyrrolidinium | | | | | |
| | methylcarbonate | | | | | |
| 3³ | 1-butyl-1- | 100° C. | 1:5 | 100 | 79 | 21 |
| | methylpyrrolidinium | | | | | |
| | methylcarbonate | | | | | |
| 4³ | 1-butyl-1- | 120° C. | 1:5 | 100 | 90 | 8 |
| | methylpyrrolidinium | | | | | |
| | methylcarbonate | | | | | |
| 5³ | 1-butyl-1- | 120° C. | 1:8 | 100 | 89 | — |
| | methylpyrrolidinium | | | | | |
| | methylcarbonate | | | | | |
| 6³ | 1-butyl-1- | 120° C. | 1:15 | 100 | 41 | 30 |
| | methylpyrrolidinium | | | | | |
| | methylcarbonate | | | | | |
| 7³ | 1-butyl-1- | 140° C. | 1:5 | 100 | 79 | 1 |
| | methylpyrrolidinium | | | | | |
| | methylcarbonate | | | | | |
| 8³ | 1-butyl-1- | 160° C. | 1:5 | 100 | 76 | 0 |
| | methylpyrrolidinium | | | | | |
| | methylcarbonate | | | | | |
| 9³ | tetramethylammonium | 100° C. | 1:5 | 100 | 79 | 0 |
| | hydroxide | | | | | |
| 10³ | tetramethylammonium | 120° C. | 1:5 | 97 | 82 | 15 |
| | hydroxide | | | | | |
| 11³ | tetramethylammonium | 140° C. | 1:5 | 100 | 65 | 21 |
| | hydroxide | | | | | |
| 12³ | tetramethylammonium | 160° C. | 1:5 | 100 | 58 | 0 |
| | hydroxide | | | | | |
| 13³ | tributylmethylammonium | 100° C. | 1:5 | 78 | 45 | 32 |
| | methylcarbonate | | | | | |
| 14³ | tributylmethylammonium | 120° C. | 1:5 | 96 | 83 | 13 |
| | methylcarbonate | | | | | |
| 15³ | tributylmethylammonium | 140° C. | 1:5 | 100 | 69 | 21 |
| | methylcarbonate | | | | | |
| 16³ | tributylmethylammonium | 160° C. | 1:5 | 100 | 39 | 0 |
| | methylcarbonate | | | | | |

¹Reaction time = 15 minutes; Heating = oil bath
²Reaction time = 30 minutes; Heating = oil bath
³Microwave heating
GL = Glycerol;
DMC = Dimethylcarbonate,
GD = Glycidol,
GC = Glycerol carbonate

The results of Table 1 show a surprisingly high rate of conversion and selectivity for glycidol achieved in step i) of the process according to the present invention and obtainable within a short reaction time. For instance, a GL conversion of 100% and a GD selectivity of 85% is obtained when GL and DMC, in a GL:DMC molar ratio of 1:5, are reacted in the presence of 1-butyl-1-methylpyrrolidinium methylcarbonate catalyst for 15 minutes at 120° C. using heat from an oil bath (Entry 1).

A GL conversion of 100% and a GD selectivity of 90% is obtained when GL and DMC, in a GL:DMC molar ratio of 1:5, are reacted in the presence of a 1-butyl-1-methylpyrrolidinium methylcarbonate catalyst at 120° C. in a microwave for a hold time of 15 minutes (Entry 4). A GL conversion of 97% and a GD selectivity of 82% is obtained when GL and DMC, in a GL:DMC molar ratio of 1:5, are reacted in the presence of a tetramethylammonium hydroxide catalyst at 120° C. in a microwave for a hold time of 15 minutes (Entry 10).

Isolation of Glycidol

The general procedure for isolating glycidol formed in the ionic liquid catalysed reactions was by liquid-liquid extraction. Specifically, two volume equivalents of ethyl acetate based on the volume of the reaction mixture and a single equivalent of water based on the volume of the reaction mixture were added to reaction vessel following completion of the reaction. Glycidol preferentially partitioned into the organic phase, which was then separated by decanting, before the organic solvent was removed under reduced pressure leaving a crude glycidol product.

Reaction of Glycidol with Amine of Formula III

Glycidol prepared from the reaction of dimethyl carbonate and glycerol in the presence of an ionic liquid was added to a vessel containing an amine and the resulting mixture stirred at elevated temperature in order to form the corresponding alkanolamine. In the following examples glycidol was used in pure form. However, it is possible to transfer the product mixture of step ii) of the process comprising glycidol and the ionic liquid catalyst directly to the amine without having to first isolate glycidol. Although less preferred, it is also possible to transfer amine to the product mixture of step ii) comprising glycidol and ionic liquid catalyst. However, this approach may increase the possibility of side undesired side reactions, such as glycidol polymerisation, and therefore reduce alkanolamine yield.

### Example 9

Alkanolamine Mixture A

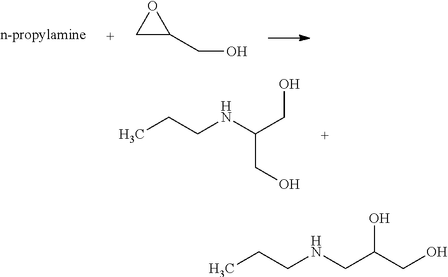

Glycidol (4.96 g, 64 mmol) was added dropwise to n-propylamine (39.9 g, 675 mmol, 10.5 equiv) at 5° C. and the mixture stirred for 16 h. After this time, the excess n-propylamine was removed under vacuum. 7.49 g|_{[MJS1]}(88.1% yield) of a colourless, slightly viscous liquid was recovered. From ¹H NMR, the products formed were a mixture of the ring-opened structural isomers, as illustrated above and conversion was complete with respect to glycidol. The amounts of each structural isomers formed could not be determined from the ¹H NMR as a result of signal overlap.

### Example 10

Alkanolamine Mixture B

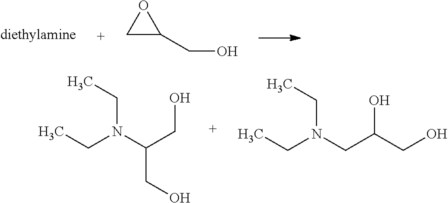

Glycidol (5.143 g, 69 mmol) was added dropwise to diethylamine (5.608 g, 77 mmol, 1.12 equiv) at 50° C. and the mixture stirred for 16 h. After this time, the excess diethylamine was removed under vacuum. 7.61 g (89.7% yield) of a colourless, slightly viscous liquid was recovered. From ¹H NMR, the products formed were a mixture of the ring-opened structural isomers, as illustrated above, and conversion was complete with respect to glycidol. The amounts of each structural isomer formed could not be determined from ¹H NMR as a result of signal overlap.

Assessing Physical Properties of Alkanolamines

### Example 11

Boiling points for alkanolamine mixtures A and B, as well as industry standards MEA, DEA and MDEA were measured at reduced pressure. Atmospheric pressure boiling points were subsequently determined by extrapolation using a nomograph. The results are presented in Table 2 below. The results show that alkanolamine mixtures A and B both have boiling points which are significantly greater than MEA, and which are comparable to the boiling points of DEA and MDEA. As discussed hereinbefore, high boiling point is advantageous for reducing losses in acid-gas desorption processes during alkanolamine recycling. Alkanolamine mixture A has a boiling point which is 30° C. higher than that of Alkanolamine mixture B. This is believed to be due to increased hydrogen bonding of the secondary alkanolamine relative to the tertiary alkanolamine.

TABLE 2

| Compound | Boiling Point (° C.) |
|---|---|
| MEA | 170.8 |
| DEA | 268.8 |
| MDEA | 246-248 |
| Alkanolamine A | 262-263 |
| Alkanolamine B | 232-233 |

### Example 12

Vapour pressures for alkaolamine mixtures A and B, as well as for MEA, DEA and MDEA were determined using an isoteniscope equipped with an Edwards pressure sensor. FIG. 4 shows the vapour pressure-temperature profiles for industry standards MEA, DEA and MDEA, from which it is clear that MEA has a significantly higher vapour pressure than DEA and MDEA. FIG. 5 shows the vapour pressure-temperature profiles for industry standards DEA and MDEA against alkanolamines A and B. FIG. 5 demonstrates that the alkanolamines A and B have comparable vapour pressure to DEA and MDEA.

CO₂ Uptake Study—Experimental Procedure

In a typical experiment, the volume of a pressure vessel [Parr pressure system] was first determined by evacuating it under reduced pressure and subsequently pumping a known amount of gas at a certain temperature and pressure into the vessel. Measurement of the amount of gas was read as the volume of gas at standard conditions from the mass flow controller [BROOKS Smart Massflow]. The ideal gas law was used to calculate the actual volume of the pressure vessel.

A known mass and volume of alkanolamine-water mixture was placed in a pressure vessel and the vessel evacuated to 10 kPa. Carbon dioxide was then pumped into the stirred pressure vessel (500 rpm) through the mass flow controller up to 500 kPa and at 20.0° C. The system is allowed to equilibrate for 1 hour, or until no more gas was being added according to the mass flow controller.

Calculation of the total amount of gas introduced into the pressure vessel is made using the reading in the mass flow controller. The actual amount of gas in the gas phase was calculated by the ideal gas law, where the volume of the gas phase was equal to the volume of the pressure vessel minus the volume of the liquid phase. The amount of gas dissolved in the liquid phase was calculated by subtracting the actual amount of gas in the gas phase from the total amount of gas introduced into the pressure vessel.

### Example 13

A 40 wt % aqueous solution of alkanolamine mixture A was prepared. 4.858 ml of the mixture was transferred to the autoclave (500 rpm), and subsequently equilibrated to 20° C. A momentary vacuum was applied, followed by the introduction of CO₂ via a mass flow controller. The reactor was pressurised to 500 kPa and the total volume of CO₂ added was recorded. The results are presented in Error! Reference source not found., as moles of CO₂ absorbed per litre, moles absorbed per kg of absorbent and moles CO₂ absorbed per mol alkanolamine.

### Example 14

A 40 wt % aqueous solution of alkanolamine mixture B was prepared. 4.923 ml of the mixture was transferred to the autoclave, and subsequently equilibrated to 20° C. A momentary vacuum was applied, followed by the introduction of CO₂ via a mass flow controller. The reactor was pressurised to 500 kPa and the total volume of CO₂ added was recorded. The results are presented in Error! Reference source not found., as moles of CO₂ absorbed per litre, moles absorbed per kg of absorbent and moles CO₂ absorbed per mol alkanolamine.

### Comparative Example 1

A 40 wt % solution of diethanolamine (DEA) in water was prepared. 4.984 ml of the mixture was transferred to the autoclave, and subsequently equilibrated to 20° C. A momentary vacuum was applied, followed by the introduction of CO₂ via a mass flow controller. The reactor was pressurised to 500 kPa and the total volume of CO₂ added was recorded. The results are presented in Error! Reference source not found., as moles of CO₂ absorbed per litre, moles absorbed per kg of absorbent and moles CO₂ absorbed per mol alkanolamine.

### Comparative Example 2

A 40 wt % solution of methyl diethanolamine (MDEA) in water was prepared. 4.887 ml of the mixture was transferred to the autoclave, and subsequently equilibrated to 20° C. A momentary vacuum was applied, followed by the introduction of CO₂ via a mass flow controller. The reactor was pressurised to 500 kPa and the total volume of CO₂ added was recorded. The results are presented in Error! Reference source not found., as moles of CO₂ absorbed per litre, moles absorbed per kg of absorbent and moles CO₂ absorbed per mol alkanolamine.

TABLE 3

| | | Vol. | CO₂ | CO₂ | CO₂/ |
|---|---|---|---|---|---|
| Alkanol- | Alkanolamine/ | added | Absorbed | Absorbed | alkanolamine |
| amine | water (w/w) | (ml) | (mol/L) | (mol/kg) | (mol/mol) |
| A | 40/60 | 4.858 | 1.98 | 1.94 | 0.64 |
| B | 40/60 | 4.923 | 1.97 | 1.93 | 0.71 |
| DEA | 40/60 | 4.984 | 3.16 | 3.01 | 0.79 |
| MDEA | 40/60 | 4.887 | 2.91 | 3.47 | 0.84 |

The results presented in Table 3 demonstrate that alkanolamines according to Formula I and II of the present invention exhibit comparable CO₂ absorption with industry standard alkanolamines, DEA and MDEA. A notable advantage of the alkanolamines according to the present invention (A and B) is that they are more cost effective to prepare from cheap and readily available glycerol precursor.

### Example 15

The methods of Examples 13 and 14 were repeated using different reactor pressures (between 100 kPa and 700 kPa CO₂ partial pressure). The same procedure was also adopted across the same range of CO₂ partial pressure for 40 wt % aqueous solution of MEA. For each CO₂ partial pressure tested, the corresponding absorption capacity, ‘a’ (mol CO₂/mol alkanolamine), of the alkanolamine absorbent was determined. The results of these experiments, together with those for Comparative Examples 1 and 2 (500 kPa CO₂ partial pressure) are represented graphically in FIG. 6. The results show that alkanolamine mixtures A and B, preparable by the process of the invention, have CO₂ absorption capacities which are comparable with industry standards MEA, DEA and MDEA.

The results of CO₂ repeated uptake experiments, performed substantially as described in Examples 13 and 14, as well as Comparative Examples 1 and 2, and conducted at a CO₂ partial pressure of 500 kPa (5 bar) for alkanolamine mixtures A and B, and industry standards MEA, DEA and MDEA, are shown in FIG. 7. FIG. 7 demonstrates a high level of reproducibility of results with alkanolamines prepared in accordance with the present invention, which is comparable to the reproducibility observed with industry standards MEA, DEA and MDEA.

Chemical and Physical Absorption

### Example 16

To observe the chemical and physical absorption behaviour of alkanolamines prepared in accordance with the process of the invention, uptake of CO₂ by a 40 wt. % aqueous mixture of Alkanolamine Mixture A was measured for varying CO₂ partial pressure.

Alkanolamine Mixture A:

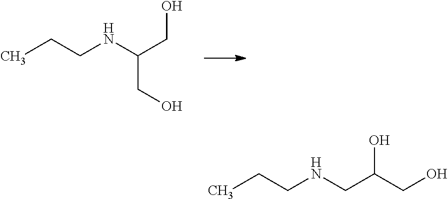

The alkanolamine solution was added to a cleaned/dried vessel and the vessel evacuated to 10 kPa (0.1 bar). CO₂ pressure was then adjusted to 100 kPa (1 bar) and CO₂ was introduced via a mass flow controller. After 2 hours, pressure was equilibrated at 100 kPa (1 bar) and the volume of CO₂ added recorded. Subsequently, pressure is increased to 150 kPa (1.5 bar) and equilibrated and increased thereafter in units of 50 kPa (0.5 bar). The results of the CO₂ solubility in the alkanolamine mixture are presented in FIG. 8. FIG. 8 clearly shows absorption of CO₂ beyond 0.5 mol CO₂ per mol of alkanolamine, as CO₂ partial pressure is increased, indicating physical absorption in addition to chemical absorption.

1. A process for preparing an alkanolamine compound of formula I and/or an alkanolamine compound of formula II, or salts thereof: 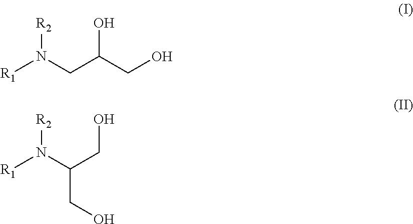
wherein: R₁ and R₂ are independently selected from hydrogen, a C₁ to C₈, straight chain or branched alkyl group, a C₂ to C₈ straight chain or branched alkenyl group, a C₃ to C₈ cycloalkyl group, a C₆ to C₁₀ aryl group, a 3 to 10 membered heterocyclic group, a polysaccharide group, a polyethylene oxide group, or R₁ and R₂ together with the nitrogen atom to which they are attached form a heterocyclic group, wherein said alkyl, alkenyl, cycloalkyl, aryl and heterocyclic groups are unsubstituted or may be substituted by one to three groups selected from: C₁ to C₆ alkoxy, C₂ to C₈ alkoxyalkoxy, C₃ to C₈ cycloalkyl, C₆ to C₁₀ aryl, 3 to 10 membered heterocyclic, C₇ to C₁₀ aralkyl, 3 to 10 membered heterocyclic-C₁ to C₄ alkyl, —OH, —CH₂CH(OH)CH₂(OH), —CH(CH₂OH)₂, or —NR₃R₄, wherein R₃ and R₄ are independently selected from hydrogen or C₁ to C₆ straight chain or branched alkyl group;
said process comprising the following steps:
i) preparing glycidol in a one-pot reaction of glycerol with dimethylcarbonate, at a temperature of from 100° C. to 160° C. and a molar ratio of glycerol to dimethylcarbonate of from 1:4 to 1:10, in the presence of an ionic liquid catalyst having the formula:
[Cat⁺][X⁻]
wherein: [Cat⁺] represents one or more cationic species, and
[X⁻] represents one or more anionic species;
ii) reacting the product of step i) with an amine of formula III, 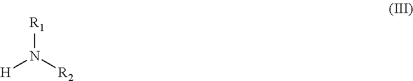
wherein: R₁ and R₂ are as defined for formula I and II.

2. A process according to claim 1, wherein the molar ratio of glycerol to dimethylcarbonate in step i) is from 1:5 to 1:8.

3. A process according to claim 1 or claim 2, wherein the reaction in step i) is conducted at a temperature of from 110° C. to 140° C.

4. A process according to any of claims 1 to 3, wherein the reaction in step i) is conducted at a temperature of from 115° C. to 130° C.

5. A process according to any of claims 1 to 4, wherein the reaction in step i) is conducted at a temperature of from 115° C. to 125° C.

6. A process according to any of claims 1 to 5, wherein the amount of ionic liquid catalyst in step i) is at least 5 mol % based on glycerol.

7. A process according to any of claims 1 to 6, wherein the glycidol formed in step i) is reacted with the amine of formula III in step ii) without prior separating from the reaction mixture.

8. A process according to any of claims 1 to 6, wherein the glycidol formed in step i) is isolated from the reaction mixture before being reacted with the amine of formula III in step ii).

9. A process according to claim 8, wherein glycidol is isolated from the reaction mixture using liquid-liquid extraction and glycidol is preferentially extracted into an organic phase.

10. A process according to claim 9, wherein the liquid extraction is with ethyl acetate and glycidol is preferentially extracted into an ethyl acetate organic phase.

11. A process according to claim 8, wherein glycidol is isolated from the reaction mixture using azeotropic distillation.

12. A process according to claim 11, wherein azeotropic distillation is performed using cumene.

13. A process according to claim 12, wherein the glycidol-cumeme mixture obtained from azeoptropic distillation is used directly for reaction with the amine of formula III in step ii).

14. A process according to any of claims 1 to 13 wherein glycidol is added portion-wise to the amine of formula III for reaction in step ii).

15. A process according to claim 14 wherein glycidol is added drop-wise to the amine of formula III for reaction in step ii).

16. A process according to any of claims 1 to 15, wherein the reaction in step ii) is conducted at a temperature of from 10° C. to 100° C.

17. A process according to any of claims 1 to 16, wherein the reaction in step ii) is conducted at a temperature of from 30° C. to 70° C.

18. A process according to any of claims 1 to 17, wherein the reaction in step ii) is conducted at a temperature of from 40° C. to 60° C., for example 50° C.

19. A process according to any of claims 1 to 18, further comprising isolating the alkanolamine compound of formula I and/or II from the product mixture of step ii).

20. A process according to claim 19, wherein the alkanolamine compound of formula I and/or II is isolated by distillation.

21. A process according to any of claims 1 to 20, wherein R₁ and R₂ are independently selected from hydrogen, a C₁ to C₈, straight chain or branched alkyl group, a C₂ to C₈ straight chain or branched alkenyl group, a C₃ to C₈ cycloalkyl group, a C₆ to C₁₀ aryl group, a 3 to 10 membered heterocyclic group, or R₁ and R₂ together with the nitrogen atom to which they are attached form a heterocyclic group, wherein said alkyl, alkenyl, cycloalkyl, aryl and heterocyclic groups are unsubstituted or may be substituted by one to three groups selected from: C₁ to C₆ alkoxy, C₂ to C₈ alkoxyalkoxy, C₃ to C₈ cycloalkyl, C₆ to C₁₀ aryl, 3 to 10 membered heterocyclic, C₇ to C₁₀ aralkyl, 3 to 10 membered heterocyclic-C₁ to C₄ alkyl, —OH, —CH₂CH(OH)CH₂(OH), —CH(CH₂OH)₂, or —NR_{y}R^{z}, wherein R_{y} and R_{z} are independently selected from hydrogen or C₁ to C₆ straight chain or branched alkyl group.

22. A process according to any of claims 1 to 21, wherein R₁ and R₂ are independently selected from hydrogen, a C₁ to C₆, straight chain or branched alkyl group, a C₃ to C₆ cycloalkyl group, a C₆ to C₁₀ aryl group, a 3 to 8 membered heterocyclic group, or R₁ and R₂ together with the nitrogen atom to which they are attached form a heterocyclic group, wherein said alkyl, cycloalkyl, aryl, heterocyclic groups are unsubstituted or may be substituted by one to three groups selected from: C₁ to C₆ alkoxy, C₂ to C₈ alkoxyalkoxy, C₃ to C₆ cycloalkyl, C₆ to C₁₀ aryl, 3 to 8 membered heterocyclic, C₇ to C₁₀ aralkyl, 3 to 10 membered heterocyclic-C₁ to C₄ alkyl, —OH, —CH₂CH(OH)CH₂(OH), —CH(CH₂OH)₂, or —NR_{y}R^{z}, wherein R_{y} and R^{z} are independently selected from hydrogen or C₁ to C₆ straight chain or branched alkyl group.

23. A process according to any of claims 1 to 22, wherein R₁ and R₂ are independently selected from hydrogen, a C₁ to C₆, straight chain or branched alkyl group, a C₃ to C₆ cycloalkyl group, a C₆ to C₁₀ aryl group, a 3 to 8 membered heterocyclic group, or R₁ and R₂ together with the nitrogen atom to which they are attached form a heterocyclic group, wherein said alkyl, cycloalkyl, aryl, heterocylic groups are unsubstituted or may be substituted by one to three groups selected from: C₁ to C₆ alkoxy, C₂ to C₈ alkoxyalkoxy, C₃ to C₆ cycloalkyl, C₆ to C₁₀ aryl, 3 to 8 membered heterocyclic, —OH, —CH₂CH(OH)CH₂(OH), —CH(CH₂OH)₂, or —NR_{y}R^{z}, wherein R_{y} and R_{z} are independently selected from hydrogen or C₁ to C₆ straight chain or branched alkyl group.

24. A process according to any of claims 1 to 23, wherein R₁ and R₂ are independently selected from hydrogen and a C₁ to C₆, straight chain or branched alkyl group, or R₁ and R₂ together with the nitrogen atom to which they are attached form a heterocyclic group, wherein said alkyl group or said heterocyclic group are unsubstituted or may be substituted by one to three groups selected from: C₁ to C₆ alkoxy, C₂ to C₈ alkoxyalkoxy, C₃ to C₆ cycloalkyl, C₆ to C₈ aryl, 3 to 8 membered heterocyclic, —OH, —CH₂CH(OH)CH₂(OH), —CH(CH₂OH)₂, or —NR_{y}R^{z}, wherein R_{y} and R_{z} are independently selected from hydrogen or C₁ to C₆ straight chain or branched alkyl group.

25. A process according to any of claims 1 to 24, wherein R₁ and R₂ are independently selected from hydrogen and a C₁ to C₆, straight chain or branched alkyl group wherein said alkyl group is unsubstituted or may be substituted by one to three groups selected from: C₃ to C₆ cycloalkyl, C₆ to C₈ aryl, —OH and —NR_{y}R^{z}, wherein R_{y} and R^{z} are independently selected from hydrogen or C₁ to C₆ straight chain or branched alkyl group.

26. A process according to any of claims 1 to 25, wherein R₁ and R₂ are independently selected from hydrogen and a C₁ to C₆, straight chain or branched alkyl group wherein said alkyl group is unsubstituted or may be substituted by one to three groups selected from: —OH and —NR_{y}R^{z}, wherein R_{y} and R^{z} are independently selected from hydrogen or C₁ to C₆ straight chain or branched alkyl group.

27. A process according to any of claims 1 to 20, wherein R₁ is selected from a polysaccharide group or a polyethylene oxide group.

28. A process according to claim 27, wherein R₁ is derived from chitosan polysaccharide.

29. A process according to any of claims 1 to 28, wherein [Cat⁺] comprises a cationic species selected from: ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium, and uronium.

30. A process according to any of claims 1 to 29 wherein [Cat⁺] comprises an acyclic cation selected from:
[N(R^{a})(R^{b})(R^{c})(R^{d})]⁺, [P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₃₀, straight chain or branched alkyl group, a C₃ to C₈ cycloalkyl group, or a C₆ to C₁₀ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

31. A process according to any of claims 1 to 30 wherein [Cat⁺] comprises an aromatic heterocyclic cationic species selected from: benzimidazolium, benzofuranium, benzothiophenium, benzotriazolium, cinnolinium, diazabicyclodecenium, diazabicyclononenium, diazabicyclo-undecenium, dithiazolium, imidazolium, indazolium, indolinium, indolium, oxazinium, oxazolium, iso-oxazolium, oxathiazolium, phthalazinium, pyrazinium, pyrazolium, pyridazinium, pyridinium, pyrimidinium, quinazolinium, quinolinium, iso-quinolinium, quinoxalinium, tetrazolium, thiadiazolium, iso-thiadiazolium, thiazinium, thiazolium, iso-thiazolium, triazinium, triazolium, and iso-triazolium.

32. A process according to any of claims 1 to 31, wherein [Cat⁺] comprises a saturated heterocyclic cation selected from cyclic ammonium, 1,4-diazabicyclo[2.2.2]octanium, morpholinium, cyclic phosphonium, piperazinium, piperidinium, quinuclidinium, and cyclic sulfonium.

33. A process according to claim 32, wherein [Cat⁺] comprises a saturated heterocyclic cation having the formula:

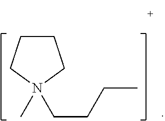

34. A process according to any of claims 1 to 33, wherein [X⁻] comprises one or more anions selected from hydroxides, halides, perhalides, pseudohalides, sulphates, sulphites, sulfonates, sulfonimides, phosphates, phosphites, phosphonates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, perchlorate, halometallates, amino acids and borates.

35. A process according to any of claims 1 to 34, wherein [X⁻] comprises an anion selected from [CO₃]²⁻, [HCO₃]⁻, [MeCO₃]⁻, [OH]⁻, and [SH]⁻.

36. A process according to any of claims 1 to 28, wherein the ionic liquid is tributylmethylammonium methylcarbonate.

37. A process according to any of claims 1 to 28, wherein the ionic liquid is 1-butyl-1-methylpyrrolidinium methylcarbonate.

38. A process according to any of claims 1 to 28, wherein the ionic liquid is tetramethylammonium hydroxide.

39. A process according to any of claims 1 to 38, further comprising the steps of:
iii) contacting a CO₂ and/or H₂S containing gaseous stream with an alkanolamine compound of formula I and/or an alkanolamine compound of formula II, or salts thereof, prepared in step ii); and
iv) obtaining a treated gaseous stream having a reduced content of CO₂ and/or H₂S compared with the CO₂ and/or H₂S containing gaseous stream of step iii).

40. A process according to claim 39, wherein the alkanolamine compound of formula I and/or II is provided in the form of a solution.

41. A process according to claim 40, wherein the total amount of alkanolamine compound of formula I and/or II in the solution is from 20% to 70% by weight.

42. A process according to claim 40 or claim 41, wherein the total amount of alkanolamine compound in the solution is from 30% to 60% by weight, for example 40% by weight.

43. A process according to any of claims 40 to 42, wherein the solution is aqueous.

44. A process according to claim 39, wherein the alkanolamine compound is provided in supported form.

45. A process according to claim 44, wherein the support is a membrane based support.

46. A process according to claim 45, wherein the support is a polyethersulfone based support.

47. A process according to any of claims 39 to 46, wherein contacting step iii) is performed at a temperature of from 10 to 80° C.

48. A process according to any of claims 39 to 47, wherein contacting step iii) is performed at a temperature of from 20 to 60° C.

49. A process according to any of claims 39 to 48, wherein contacting step iii) is performed at a temperature of from 30 to 50° C.

50. A process according to any of claims 39 to 49, wherein contacting step iii) is performed at a pressure of from 100 to 2000 kPa.

51. A process according to any of claims 39 to 50, wherein contacting step iii) is performed at a pressure of from 200 to 1000 kPa.

52. A process according to any of claims 39 to 51, wherein contacting step iii) is performed at a pressure of 500 kPa.

53. A process according to any of claims 39 to 52, further comprising separating CO₂ and/or H₂S absorbed by the alkanolamine compound of formula I and/or II in contacting step iii) by subjecting to a reduced pressure.

54. A process according to any of claims 39 to 53, further comprising separating CO₂ and/or H₂S absorbed by the alkanolamine compound of formula I and/or II in contacting step iii) by stripping with steam.

55. Use of an alkanolamine compound of formula I and/or formula II prepared by the process defined in any of the preceding claims for removing CO₂ and/or H₂S from a CO₂ and/or H₂S containing gaseous stream.

56. Use according to claim 55, wherein the gaseous stream is a methane-containing gaseous stream.

57. Use according to claim 55 or claim 56, wherein the gaseous stream is a natural gas stream.

58. Use according to claim 55, wherein the gaseous stream is a biogas-derived stream.

59. Use according to claim 55, wherein the gaseous stream is a flue gas stream.

60. Use according to claim 55, wherein the gaseous stream is a breathing gas stream for a life support system.
